(19) United States
(12) Patent Application Publication
(10) Pub. No.: US 2024/0370996 A1
(43) Pub. Date: Nov. 7, 2024

(54) SYSTEM, METHOD AND/OR COMPUTER-READABLE MEDIUM FOR MAPPING AND DISPLAYING ANATOMICAL STRUCTURES IN A USER-FRIENDLY MANNER

(71) Applicant: VENTRIPOINT DIAGNOSTICS LTD., Toronto (CA)

(72) Inventors: Desmond HIRSON, Toronto (CA); Mark SIRKIN, Toronto (CA); Claudio IRRGANG, Toronto (CA); Alvira MACANOVIC, Toronto (CA)

(73) Assignee: VENTRIPOINT DIAGNOSTICS LTD., Toronto, ON (CA)

(21) Appl. No.: 18/292,632

(22) PCT Filed: Jul. 30, 2021
(86) PCT No.: PCT/CA2021/051078

§ 371 Date: Jan. 26, 2024

(51) Int. Cl. G06T 7/00; G06T 11/00

(52) U.S. Cl. CPC G06T 7/0012; G06T 11/008; G06T 2207/10088; G06T 2207/30048

(57) ABSTRACT

The present disclosure relates generally to a system, method and/or computer readable medium for mapping anatomical structures (e.g. heart). In particular, the present disclosure relates to a system, method and/or computer readable medium for using two-and three-dimensional (2D and 3D) echocardiography (2D and 3D echo) data for mapping and displaying the anatomical structure and configuration of an organ, such as the heart.

### CROSS-REFERENCE TO RELATED APPLICATIONS

This application is a national phase entry of PCT Patent Application Serial No. PCT/CA2021/051078, filed on Jul. 30, 2021, which is incorporated by reference herein.

### TECHNICAL FIELD

The present invention relates generally to systems, methods and/or computer readable medium for mapping and displaying anatomical structures (e.g. the heart and components thereof) for the ease of the user. In particular, the present invention relates to systems, methods and/or computer readable medium for using two-and three-dimensional (2D and 3D) echocardiography (2D and 3D echo) data for mapping and displaying the anatomical structure and configuration of an organ, such as the heart.

### BACKGROUND

In the field of diagnostic imaging, the ability to precisely determine information associated with a diagnostic image (e.g. a two or three-dimensional echocardiogram, MRI, etc.) may be useful for visualizing and measuring features of anatomic structures of an organ in a body. For example, it may be desirable to use the position information associated with multiple diagnostic images to create a three-dimensional model of a desired anatomic structure, such as, for example, the heart, using techniques like 2D and/or 3D echocardiography (e.g. 2D or 3D “echo”); 3D echo is an emerging technology that is in the early stages of adoption and is heavily promoted by the major ultrasound companies. Numerous studies have demonstrated the utility of 3D over 2D echo in assessment of the left ventricular (LV) and right ventricular (RV) volume and function as well as many other structures. Magnetic Resonance Imaging (“MRI”) derived data is by its very nature presented as a 3D dataset and is used in generally the same manner as the echo-based 3D datasets.

3D echocardiogram image data provides tightly spaced images that are acquired to generate a solid volume of image data. To perform quantitative analysis, the image data may be adjusted (e.g. along the planes to select the correct 2D image) by cutting or “slicing” along one or more planes (e.g. the XY, YZ and/or XZ) into a series of parallel and/or perpendicular images along those planes. In order to examine the functioning of an organ, such as a heart, a user often “traces” the structural contours in each image to determine structural and function elements within these “slices”. Such three-dimensional echocardiogram image data is available on a number of commercial systems marketed by GE, Philips (Real-Time 3D™) and others; said systems may contain software for creating, displaying and manipulating the image data.

As described in U.S. Pat. No. 5,889,524, incorporated herein by reference, there are provided methods (e.g., KBR methodology) for reconstructing a surface of an object using three-dimensional imaging data obtained from 3D echo techniques. Digital imaging data obtained from organs (e.g. the 2D “slices” of heart) can be traced producing a data set of specific data “points” or “co-ordinates” that note, mark, define and/or correspond to a border and/or specific anatomic features or structures thereof (e.g. structural points or co-ordinates within the left ventricle (LV)). Using such structural data points, co-ordinates or points, an abstract model of a generalized structure may be generated that fits a wide range of sizes and shapes of these features or structures. The abstract model (e.g. a “template” or “overlay”) may include an abstract control mesh in which the anatomic features are labelled and sharp (edge) characteristics are identified. Coordinates can be assigned to the abstract control mesh, producing an initial embedded and subdivided mesh. The embedded subdivided mesh is rigidly aligned with the data set points of the organ's structures/features, and in particular, with the anatomic features. Finally, the aligned subdivided mesh is optimally fit to the data set points and anatomic features, yielding the reconstructed feature or structure. In applying these abstract models to medical image data, it is desirable for a user to review a number of slices and select those slices that are best for use in the abstract model. However, the selection process can be difficult and time consuming

In the prior art, such techniques may have been used to construct three-dimensional images of anatomic structures. Different companies such as TomTec Imaging Systems GMBH, Siemens, Philips and GE] have products that determine volumes in different ways. MRI measurement solutions may be AI driven (e.g. CIRCLE CARDIOVASCULAR IMAGING, Calgary) but none use the same interface and workflow across the board for all data acquisition modalities. Unfortunately, current systems fail to allow the user to easily and quickly make suitable slice selection, which may add time to the analytic process.

Tools that enhance the diagnostic value of medical imaging techniques as well as enable the diagnosis in an economically reasonable time are required to assist medical practitioners. As a result, there may be a need for, or it may be desirable to provide, a system, method, computer readable media, and/or cooperating environment that overcomes one or more of the limitations associated with the prior art. It may be advantageous to provide a system, method and/or computer readable medium for allowing for single workspace and workflow regardless of how the image data is obtained.

### SUMMARY

According to the invention, there is disclosed a system for a single workspace and workflow regardless of how the image data is obtained.

The present invention provides for method for using a computer to reconstructing anatomical features or an organ, comprising the steps of: (a) obtaining 3D image data from imaging the organ; (b) obtaining a 2D slice of the organ from the 3D image data and optimizing fit of a template having predetermined landmark points to the 2D slice so that a plurality of anatomical features of the 2D slice are generally aligned with the corresponding landmark points of the template; (c) assigning each point of the template to the corresponding anatomical feature of the 2D slice based on the fit of the template; and (d) repeating steps (b) and (c) to produce a functional reconstruction of the organ.

Another aspect of the present invention is directed to the above noted method wherein the 3D image data is echo-based or MRI 3D datasets.

Another aspect of the present invention is directed to the above noted method wherein the organ is a heart.

Yet another aspect of the present invention is directed to the above noted method wherein step (d) employs a KBR algorithm.

Yet another aspect of the present invention is directed to the above noted method wherein the templates are selected based on the anatomical regions of the heart.

Yet another aspect of the present invention is directed to the above noted method wherein the KBR algorithm functionally reconstructs the Left Ventricle (LV), Right Ventricle (RV), Left Atrium (LA), and Right Atrium (RA) of the using the assigned points of the template.

Yet another aspect of the present invention is directed to the above noted method wherein each point of the template is assigned automatically.

Yet another aspect of the present invention is directed to the above noted method wherein each point of the template is assigned by the user.

Yet another aspect of the present invention is directed to the above noted method wherein the template corresponds to anatomic region of the heart.

Yet another aspect of the present invention is directed to the above noted method wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

Yet another aspect of the present invention is directed to a system for producing a functional reconstruction of the organ, comprising: (a) an imaging system for producing 3D image data of the organ; (b) a memory for storing: (c) the 3D image data; and (d) machine instructions that define steps for processing the data derived from the 3D image data; and a processor that is coupled to the memory, said processor executing the machine instructions, causing the processor to: (i) obtain a 2D slice of the organ from the 3D image data and optimizing fit of the template to the 2D slice so that a plurality of anatomical features of the 2D slice are generally aligned with the corresponding points of the template; (ii) assign each point of the template to the corresponding anatomical feature of the 2D slice based on the fit of the template; and (iii) repeating steps (ii) and (iii) to produce a functional reconstruction of the organ.

Yet another aspect of the present invention is directed to the above noted system wherein the 3D image data is echo-based or MRI 3D datasets.

Yet another aspect of the present invention is directed to the above noted system wherein the organ is a heart.

Yet another aspect of the present invention is directed to the above noted system wherein The system of clam 13 wherein step (c) (iii) employs a KBR algorithm.

Yet another aspect of the present invention is directed to the above noted system wherein The system of claim 14 wherein the templates are selected based on the anatomical regions of the heart.

Yet another aspect of the present invention is directed to the above noted system wherein the KBR algorithm functionally reconstructs the Left Ventricle (LV), Right Ventricle (RV), Left Atrium (LA), and Right Atrium (RA) of the using the assigned points of the template.

Yet another aspect of the present invention is directed to the above noted system wherein each point of the template is assigned automatically.

Yet another aspect of the present invention is directed to the above noted system wherein herein each point of the template is assigned by the user.

Yet another aspect of the present invention is directed to the above noted system wherein the template corresponds to anatomic region of the heart.

Yet another aspect of the present invention is directed to the above noted system wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

Yet another aspect of the present invention is directed to a method for calculating, analyzing and displaying anatomical features or an organ, comprising the steps of: (a) selecting 3D image data from imaging the organ; (b) obtaining a plurality of 2D slices of the organ from the 3D image data, the plurality of 2D slices corresponding to one of the XY, YZ and/or XZ plane of the organ; and dynamically displaying the plurality of 2D slices in one of the XY, YZ and XZ planes orthogonally and having a user view the plurality of 2D slices in each of the XY, YZ or XZ planes

Yet another aspect of the present invention is directed to the above noted method wherein the 3D image data is echo-based or MRI 3D datasets.

Yet another aspect of the present invention is directed to the above noted method wherein the organ is a heart.

Yet another aspect of the present invention is directed to the above noted method wherein the system further optimizing fit of a template to the 2D slice so that a plurality of anatomical features of the 2D slice are generally aligned with the corresponding points of the template.

Yet another aspect of the present invention is directed to the above noted method wherein the templates are selected based on the anatomical regions of the heart.

Yet another aspect of the present invention is directed to the above noted method wherein each point of the template is assigned automatically.

Yet another aspect of the present invention is directed to the above noted method wherein each point of the template is assigned by the user.

Yet another aspect of the present invention is directed to the above noted method wherein the template corresponds to anatomic region of the heart.

Yet another aspect of the present invention is directed to the above noted system wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

Yet another aspect of the present invention is directed to a method for using a computer to reconstructing anatomical features or an organ, comprising the step of creating a 2D slice having predetermined landmark points generally corresponding with selected anatomical features of the organ from 3D image data.

Yet another aspect of the present invention is directed to the above noted method wherein the 3D image data is echo-based or MRI 3D datasets.

Yet another aspect of the present invention is directed to the above noted method wherein the organ is a heart.

Yet another aspect of the present invention is directed to the above noted method wherein the templates are selected based on the anatomical regions of the heart.

Yet another aspect of the present invention is directed to the above noted method wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

Yet another aspect of the present invention is directed to a graphical user interface on an electronic device with a screen display, the graphic user interface comprising: (a) a pointer displayed on the screen display, the movements of the pointer on the display screen controlled by a user; (b) a first area of the screen displaying a plurality of 2D slices of the organ obtained from 3D image data from imaging an organ, the plurality of 2D slices corresponding to one of the XY, YZ and/or XZ plane of the organ; (c) a second area of the screen separate from the first area that dynamically displays the plurality of 2D slices in each of the XY, YZ and XZ planes orthogonally; wherein based on the movements of the pointer the user can change the plurality of 2D slices that are displayed.

Yet another aspect of the present invention is directed to the above noted interface wherein the 3D image data is echo-based or MRI 3D datasets.

Yet another aspect of the present invention is directed to the above noted interface wherein the organ is a heart.

Other advantages, features and characteristics of the present invention, as well as methods of operation and functions of the related elements of the system, method and computer readable medium, and the combination of steps, parts and economies of manufacture, will become more apparent upon consideration of the following detailed description and the appended claims with reference to the accompanying drawings, the latter of which are briefly described herein below.

### BRIEF DESCRIPTION OF THE DRAWINGS

The novel features which are believed to be characteristic of the system, method and computer readable medium according to the present invention, as to their structure, organization, use, and method of operation, together with further objectives and advantages thereof, may be better understood from the following drawings in which presently preferred embodiments of the invention may now be illustrated by way of example. It is expressly understood, however, that the

FIGS. 1A to 1F are diagrams of embodiments of the present invention.

FIG. 2 is a set of diagrams of embodiments of the present invention.

FIGS. 3A and 3B are diagrams of embodiments of the present invention.

FIGS. 4A to 4Y are diagrams of embodiments of the present invention.

FIGS. 5A to 5V are diagrams of embodiments of the present invention.

FIGS. 6A to 6D are diagrams of embodiments of the present invention.

FIGS. 7A to 7H are diagrams of embodiments of the present invention.

FIGS. 8A to 8H are diagrams of embodiments of the present invention.

FIG. 9 is a diagram of embodiments of the present invention.

FIGS. 10 to 13 are diagrams of embodiments of the present invention.

### DETAILED DESCRIPTION

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order to more clearly depict certain embodiments and features of the invention.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

The present disclosure may be now described in terms of an exemplary system in which the present disclosure, in various embodiments, would be implemented. This may be for convenience only and may be not intended to limit the application of the present disclosure. It may be apparent to one skilled in the relevant art(s) how to implement the present disclosure in alternative embodiments.

In this disclosure, a number of terms and abbreviations may be used. The following definitions and descriptions of such terms and abbreviations are provided in greater detail.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

In the description and drawings herein, and unless noted otherwise, the terms “vertical”, “lateral” and “horizontal”, are generally references to a Cartesian co-ordinate system in which the vertical direction generally extends in an “up and down” orientation from bottom to top (y-axis) while the lateral direction generally extends in a “left to right” or “side to side” orientation (x-axis). In addition, the horizontal direction extends in a “front to back” orientation and can extend in an orientation that may extend out from or into the page (z-axis). A person skilled in the relevant art will also understand that the “XY plane” (e.g. XY) is the plane that contains the x and the y axis, while the YZ plane contains the y and the z axis; and the XZ plane contains the x and z axes.

It should also be appreciated that the present invention can be implemented in numerous ways, including as a method, a system, or a computer readable medium such as a computer readable storage medium or a computer network wherein program instructions are sent over a network (e.g., optical or electronic communication links). In this specification, these implementations, or any other form that the invention may take, may be referred to as processes. In general, the order of the steps of the disclosed processes may be altered within the scope of the invention.

Preferred embodiments of the present invention can be implemented in numerous configurations depending on implementation choices based upon the principles described herein. Various specific aspects are disclosed, which are illustrative embodiments not to be construed as limiting the scope of the disclosure. Although the present specification describes components and functions implemented in the embodiments with reference to standards and protocols known to a person skilled in the art, the present disclosures as well as the embodiments of the present invention are not limited to any specific standard or protocol. Accordingly, replacement standards and protocols having the same functions are considered equivalents.

A person skilled in the relevant art may generally understand a web-based application refers to any program that is accessed over a network connection using HTTP, rather than existing within a device's memory. Web-based or other applications often run inside a dashboard, browser or portal. Such applications also may be client-based, where a small part of the program is downloaded to a user's desktop or device (e.g. mobile device), but processing is done on an external server. Web-based or other applications may also be dedicated programs installed on a networked or stand-alone device, such as a smart phone, tablet or laptop. The dashboard, browser or portal is most often one specially designed site or application that brings information together from diverse sources in a uniform way. Usually, each information source gets its dedicated area on the page for displaying information (a portlet); often, the user can configure which ones to display. Portals typically provide an opportunity for users to select and/or input information into a system.

Elements of the present invention may be implemented with computer systems which are well known in the art. Generally speaking, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

The operation of an electronic device may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention may implement Knowledge-Based Reconstruction (“KBR”) algorithms (described in U.S. Pat. No. 5,889,524, incorporated herein by reference). Persons skilled in the relevant art may appreciate that KBR is a semiautomatic analytical tool that uses a database comprising knowledge of the shapes of various anatomic structures in the body, including the shapes of the anatomic structures in various disease states. KBR is based on a piecewise smooth subdivision surface reconstruction method using a sparse input of points (i.e., not whole borders) determined by a user. In an embodiment of the present invention, the user may preferably, but need not necessarily, select the images for which each part of the anatomic structure of interest is best visualized. KBR preferably does not require tracing of whole borders. Persons skilled in the art may appreciate that border tracing is difficult because images do not always show the entire border clearly (e.g., fuzzy and/or hard to identify). Border tracing is labour-intensive, and users may not trace the optimal number of borders required to model the shape and/or determine the volume of the desired anatomic structure. The measurements are preferably, but need not necessarily, made using data (e.g. an image) generated by either a two-dimensional ultrasound, three-dimensional ultrasound or magnetic resonance imaging device. To perform KBR, a user is preferably but need not necessarily only required to trace a few points on the images to mark the position of anatomic landmarks. In a preferred embodiment, a KBR algorithm utilizes knowledge concerning the shape of the organ (e.g. human heart). In a preferred embodiment, knowledge of the three-dimensional heart size and shape is used to reduce the workload that a human would typically need to accurately measure how well a patient's heart is functioning. KBR is fast as it takes about two-three minutes per volume measurement. The user preferably, but need not necessarily, provides only a very sparse input of points (i.e., not whole borders). The user can choose the highest quality images to trace those points. In other words, the user is free to work just on the images where each part of the ventricle is best seen. KBR leverages the accuracy achieved from the sparse input by utilizing a knowledge database. The database embodies knowledge of the shape of the right ventricle and how much that shape varies in human disease. The knowledge database constrains the software to produce heart-like reconstructions and to prevent the possible generation of strangely shaped surfaces.

Embodiments of the present invention may implement Artificial Intelligence (“AI”) or machine learning (“ML”) algorithms. AI and ML algorithms are general classes of algorithms used by a computer to recognize patterns, and may include on or more of the following individual algorithms: nearest neighbor, naive Bayes, decision trees, linear regression, principle component analysis (“PCA”), support vector machines (“SVM”), evolutionary algorithms, and neural networks. These algorithms may “learn” or associate patterns with certain responses in several fashions, including supervised learning, unsupervised learning, semi-supervised learning, and reinforcement learning.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following description, it is appreciated that throughout the present invention, references utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

The present invention is contemplated for use in association with one or more cooperating environments, to afford increased functionality and/or advantageous utilities in association with same. The invention, however, is not so limited.

Naturally, in view of the teachings and disclosures herein, persons having ordinary skill in the art may appreciate that alternate designs and/or embodiments of the invention may be possible (e.g., with substitution of one or more steps, algorithms, processes, features, structures, parts, components, modules, utilities, etc. for others, with alternate relations and/or configurations of steps, algorithms, processes, features, structures, parts, components, modules, utilities, etc).

Although some of the steps, algorithms, processes, features, structures, parts, components, modules, utilities, relations, configurations, etc. according to the invention are not specifically referenced in association with one another, they may be used, and/or adapted for use, in association therewith.

In certain implementations, instructions may include instructions for the analysis of image data, position data and/or reference points. While computer-readable storage medium may be a single medium, the term “computer-readable storage medium” should be taken to include a single medium or multiple media (e.g., a centralized or distributed database, and/or associated caches and servers) that store the one or more sets of instructions. The term “computer-readable storage medium” can also be taken to include any medium that is capable of storing, encoding or carrying a set of instructions for execution by the machine and that cause the machine to perform any one or more of the methodologies of the present disclosure. The term “computer-readable storage medium” can accordingly be taken to include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and software components, or only in software.

It will be understood that in establishing a user interface, a task bar may be preferably positioned at the top of a screen to provide a user interface. Preferably, a textual representation of a task's name is presented in this user interface, preferably as a button, and the task names may be shortened as necessary if display space of the button is constrained. The labelled button having the task's name preferably operate as a type of hyperlink, whereby the user/viewer can immediately switch to the activity, view, etc. of an each of the tasks by selecting the button containing the applicable name from the task bar. In other words, the user or viewer is redirected by the application to that the function represented by the task button by selecting the labelled hyperlink. Preferably, the task entry associated with the currently displayed work unit view may be shown in a different graphical representation (e.g., using a different color, font, or highlighting). In preferred embodiments, there may be provided a display having a selectable “X” in the task bar entry for each task: if the user clicks on the “X”, then its associated task may be ended and the view of its work unit may be removed. A user interface may be web-based, application based, or a combination.

The present disclosure provides a system, method and/or computer readable medium for integrating the display and manipulation of medical imaging data for the ease of the user. In a preferred embodiment of the present invention, the user seeks to capture certain 2D slices that can be matched with a guide for each image of the present invention. When reviewing 2D slices, the user can use the guide image as a means for determining whether or not to select a 2D slice for further review and processing. However, it may be difficult for the user to identify and select the relevant 2D slices that may be used for matching to the preferred guide image. This may limit the usefulness of the image data and make further analysis of the image data more difficult and time consuming. The image guides of the present invention aid the user in selecting the 2D slices from a 3D echo model. In a preferred embodiment, the image guides of the present invention allow for quick “point” placement on the 2D slices as described herein.

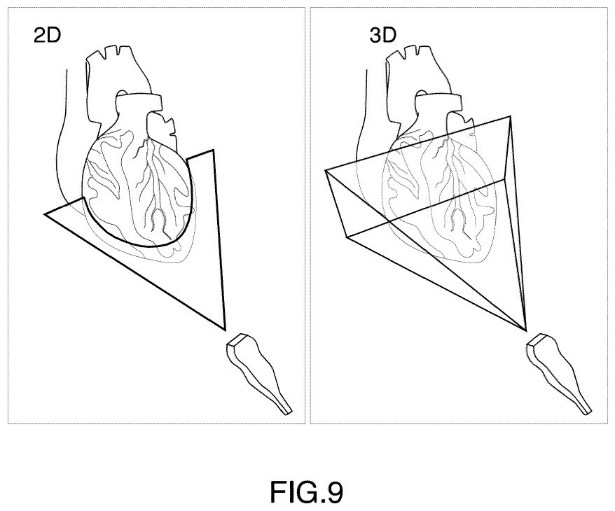

As shown in FIG. 9, preferred embodiment of the present invention permits user to obtain multiple slices in multiple planes from existing image datasets generated by any modality that images the cardiac cycle of the heart (e.g. any 3D echo device) that, in turn, can be used in further image processing (e.g. KBR techniques to accurately determine organ function characteristics such as volumetric measurements of all four chambers of the heart). In a preferred embodiment, datasets may conform to the DICOM™ 3.0 standard (Digital Imaging and Communications in Medicine), a universal format for picture archiving and communication systems (“PACS”) used in medical imaging. In a preferred embodiment of the present invention, DICOM™ compliant image datasets interact with the CLEARCANVAS™ open-source code base for medical imaging, which allows a suitable device to be integrated into the echo workflow. In a preferred embodiment, CLEARCANVAS™ forms a basis of implementation for all PACS/DICOM functionality described herein including (but not limited to) conversion from and to DICOM format and any communications involving DICOM datasets. The embodiments of the present invention use CLEARCANVAS to pack and unpack the image data obtained to conform to the internal data structure of the present invention. Systems for obtaining the medical image data, including commercial systems marketed by GE and Philips (Real-Time 3DT™) typically contain software for creating, displaying and manipulating the image data. In a preferred embodiment, the present invention incorporates or is incorporated into software to allow for such functionality, such as for example, the Ventripoint Medical System Plus (the “VMS+30” or “VMS+software”). To allow the preferred aspects of the present invention to function within such commercially available systems, there may be provided a “shell” that may be embedded into such commercial systems and apparatus. In a preferred embodiment, such a shell comprises software that exposes the operating system's services to the user or another program. In general, such operating system shells use either a command-line interface (CLI) or graphical user interface (GUI), depending on a computer's role and operation. GUIs may be preferred as they are typically designed to be easy to use. In a preferred embodiment, it is this shell that allows communication with the software built into the existing commercial systems. The medical image data is sent and received by this shell.

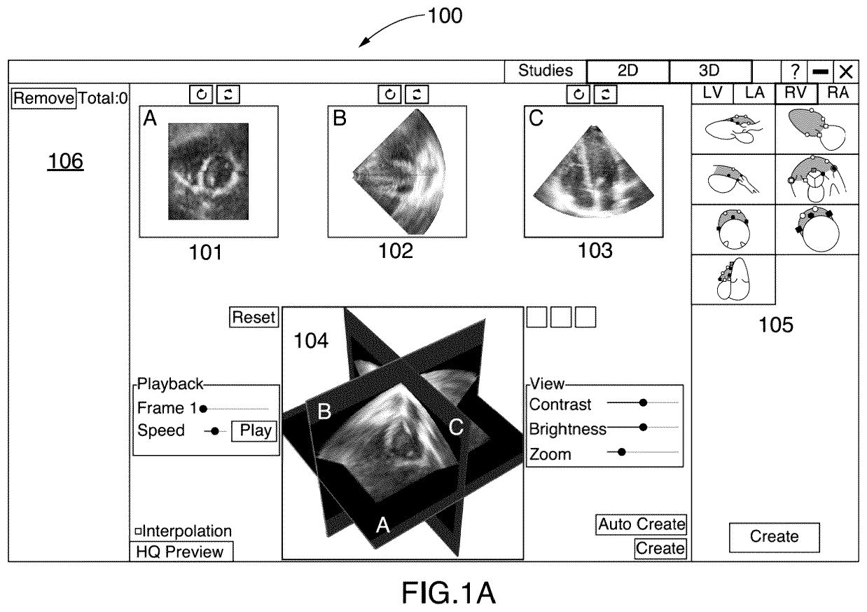

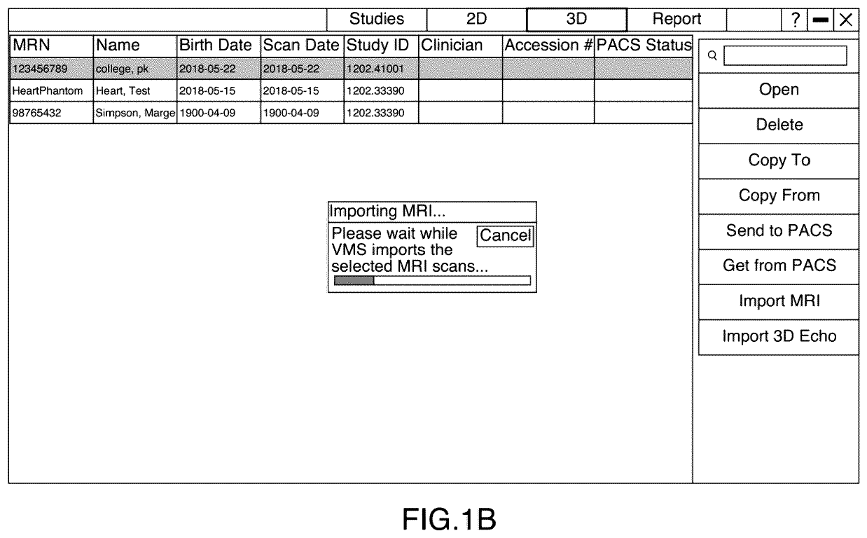

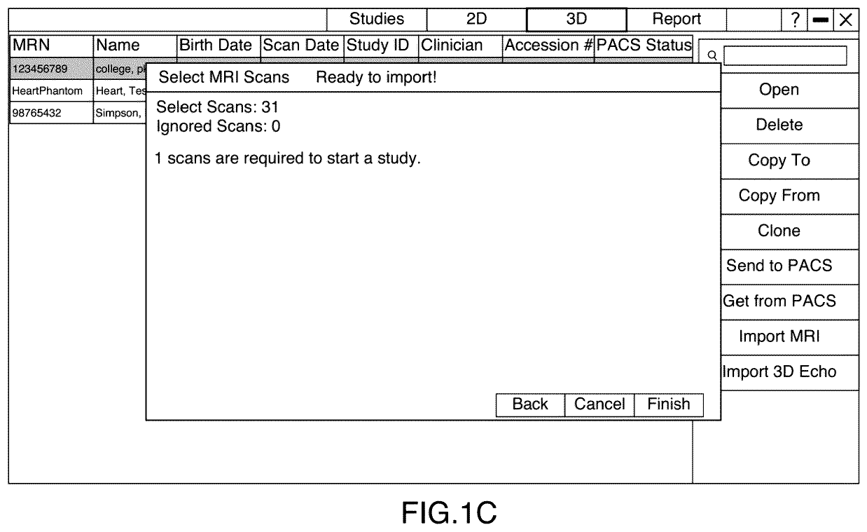

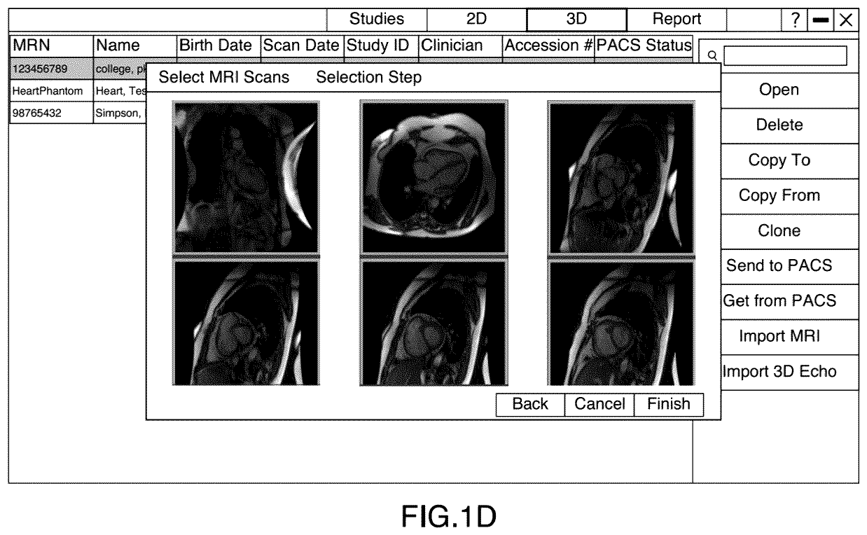

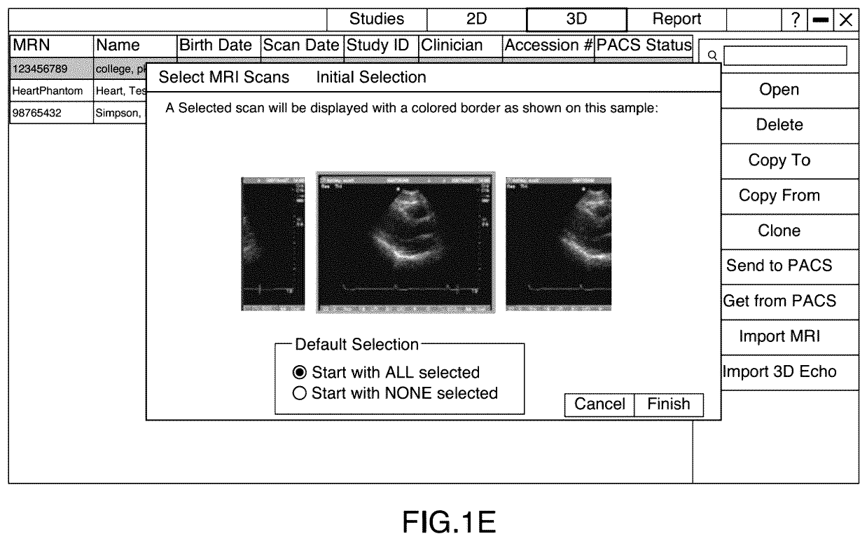

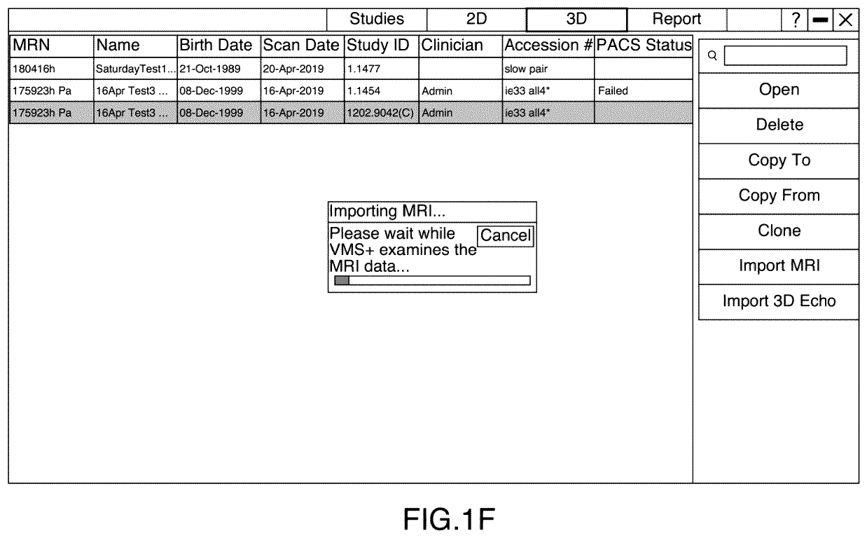

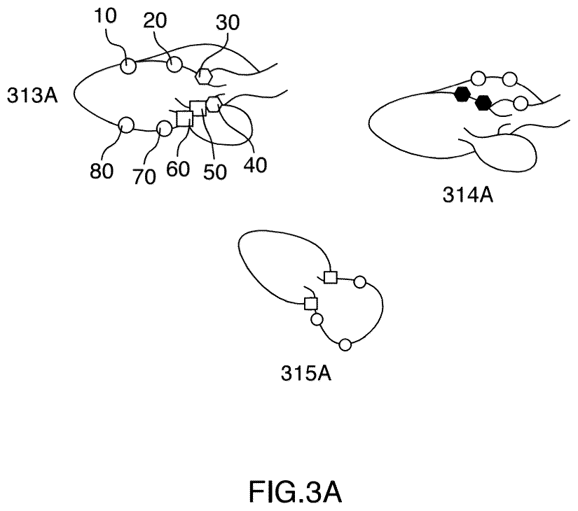

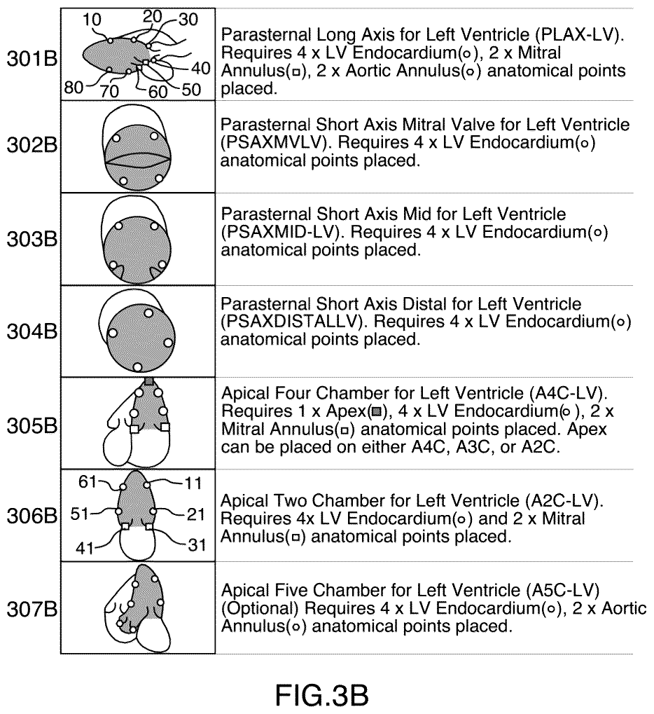

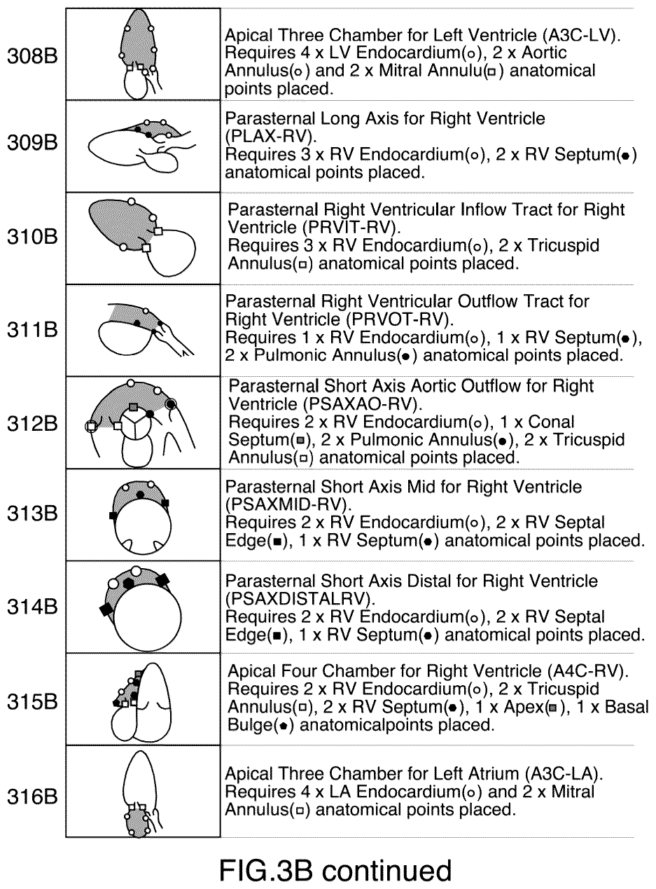

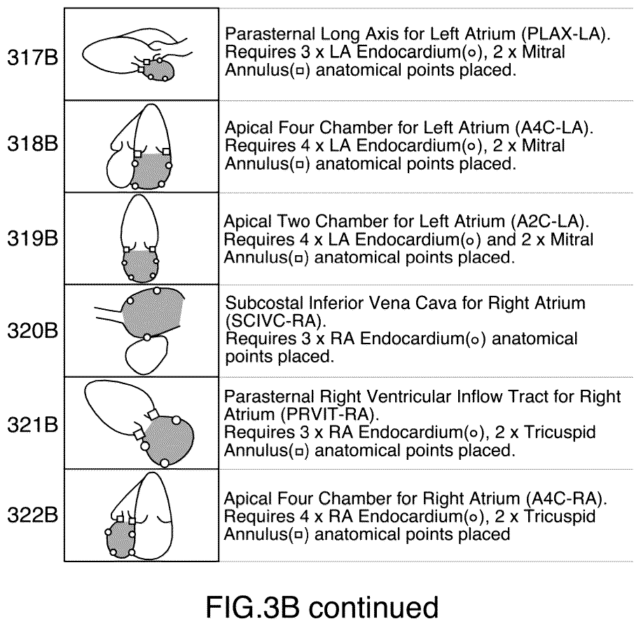

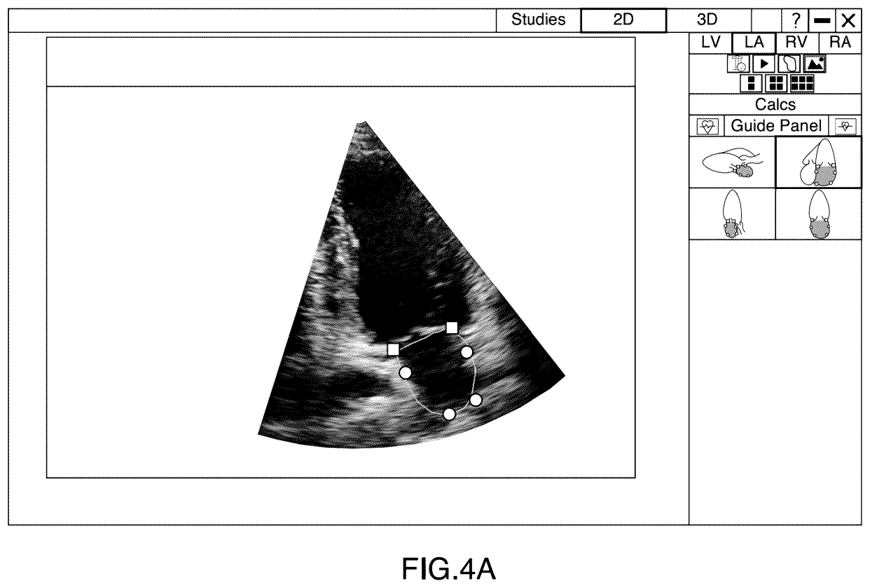

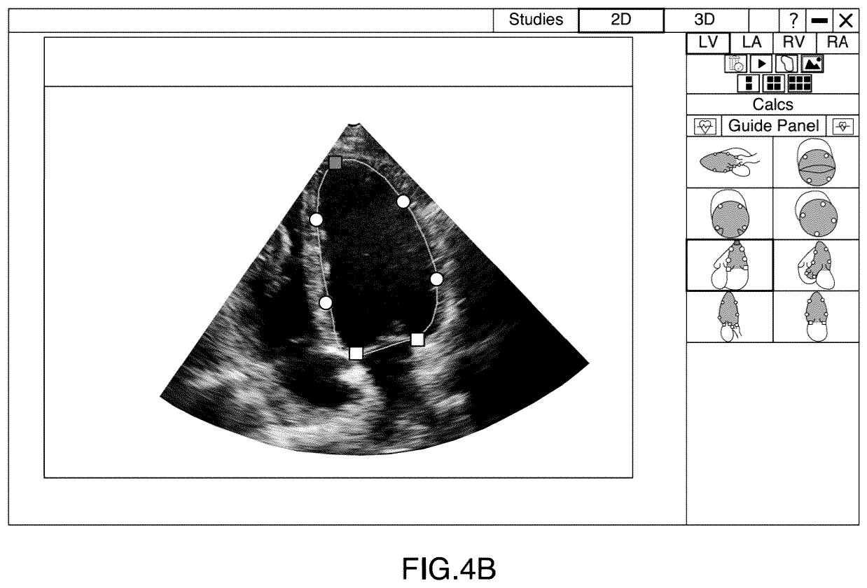

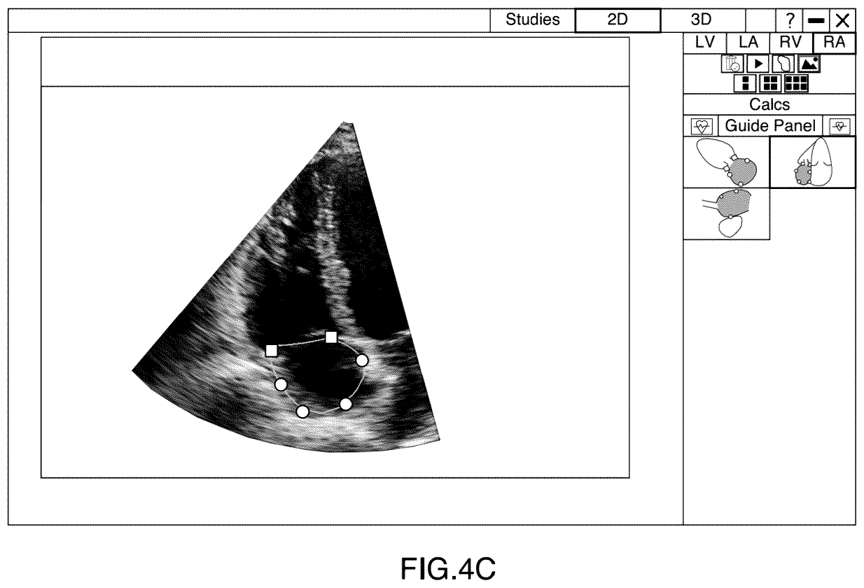

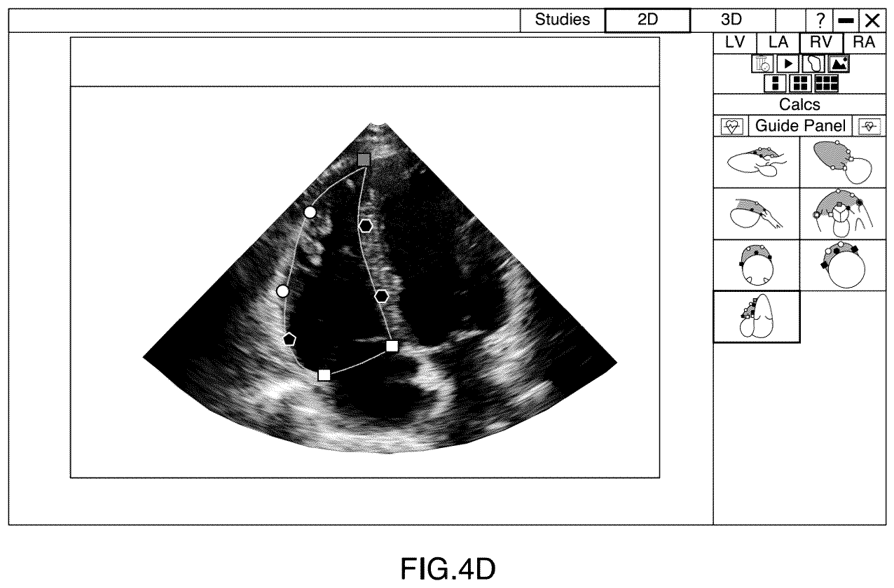

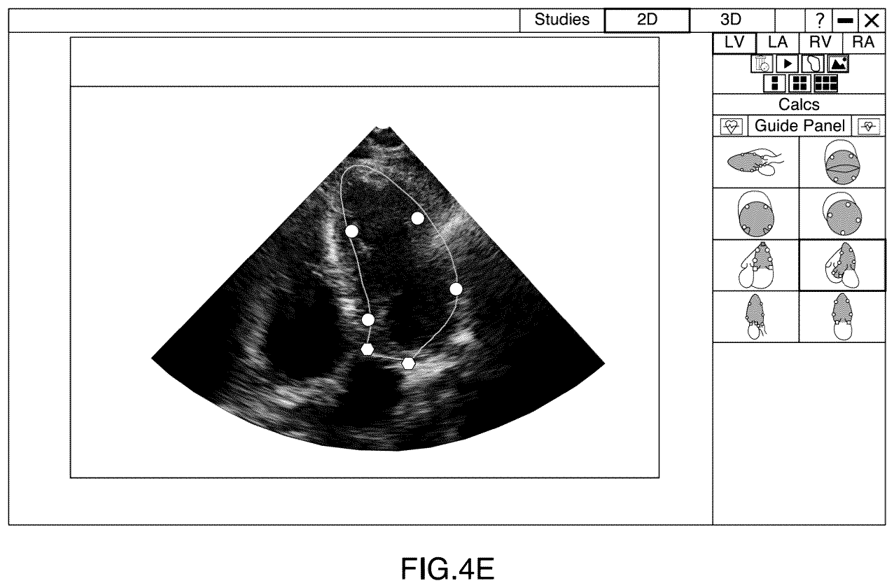

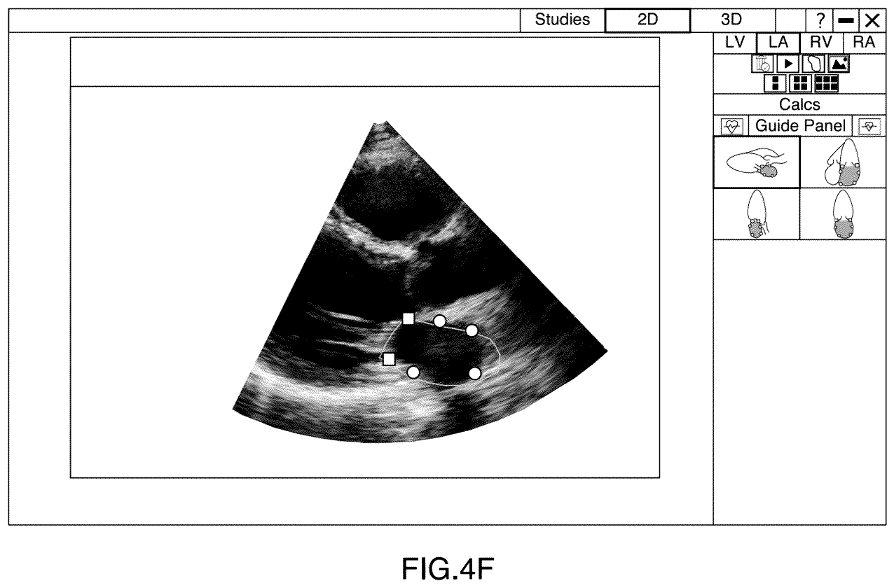

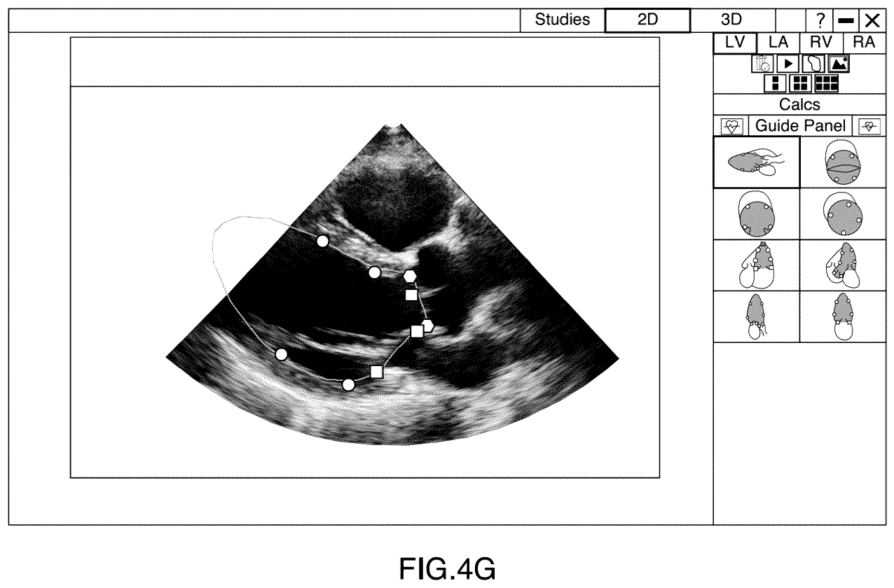

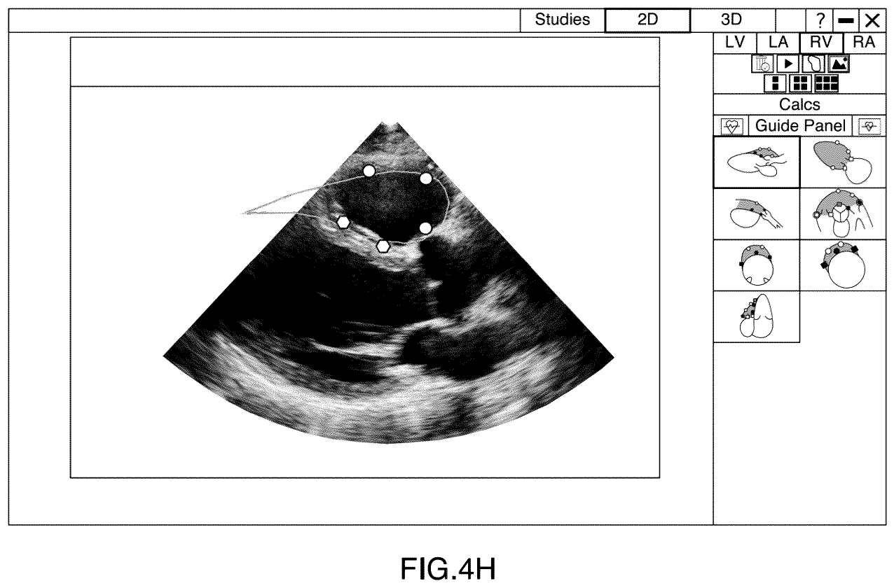

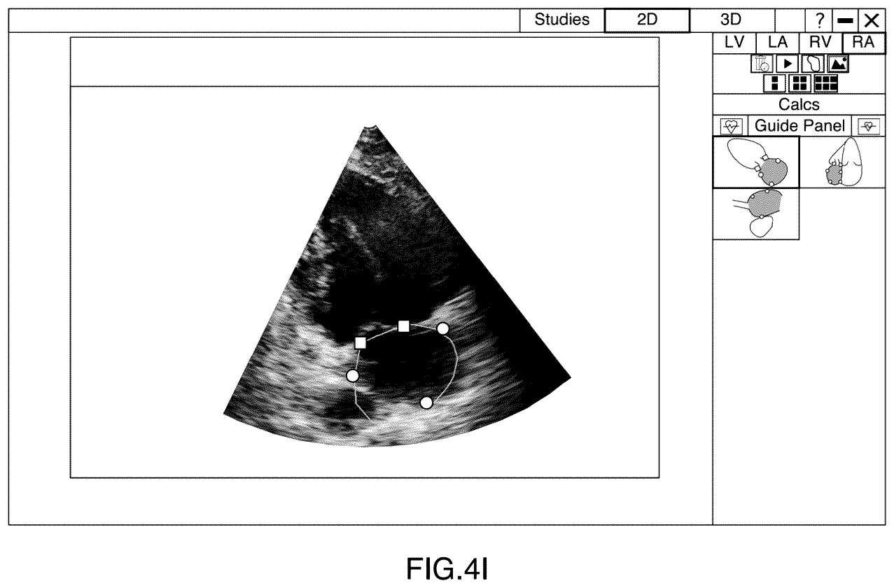

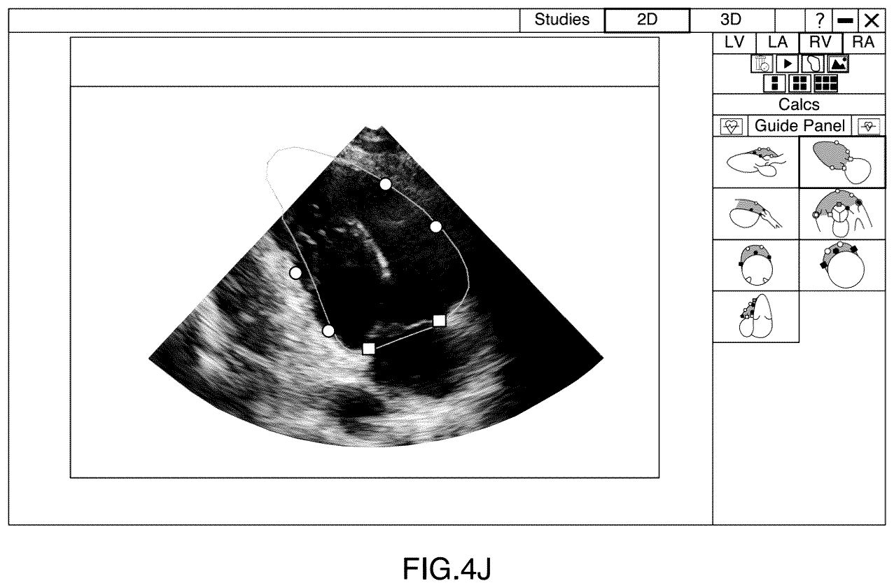

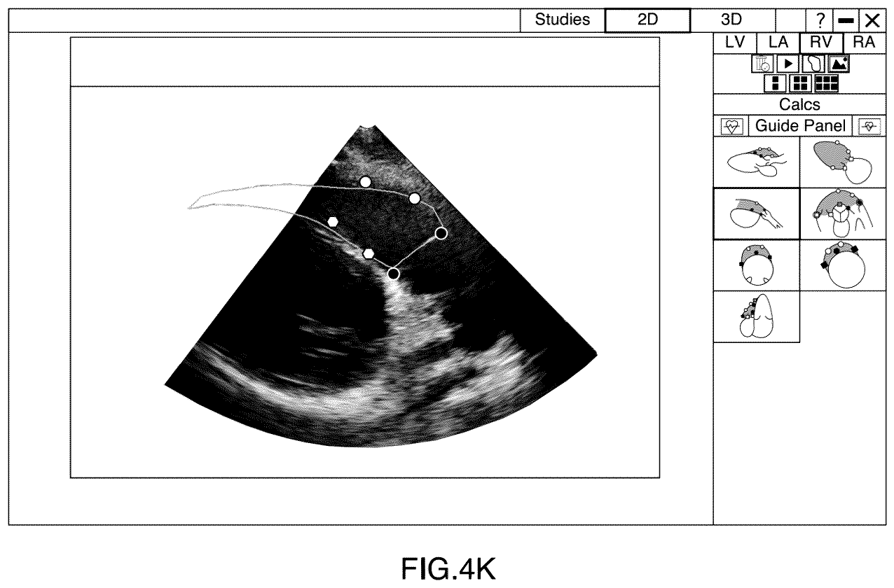

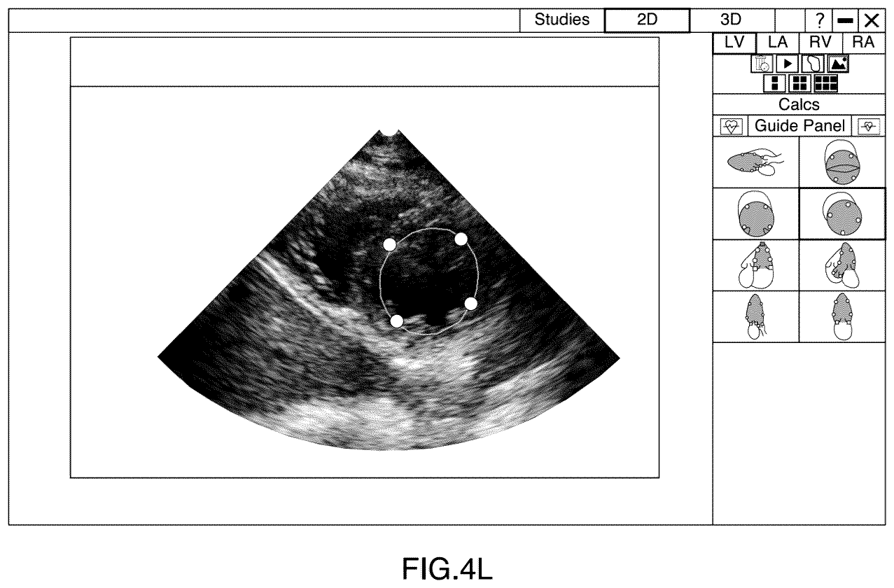

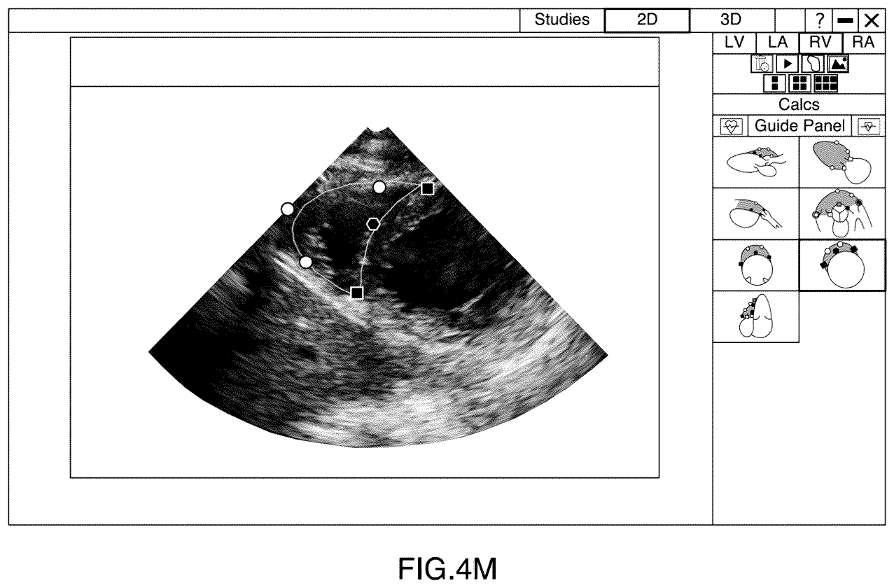

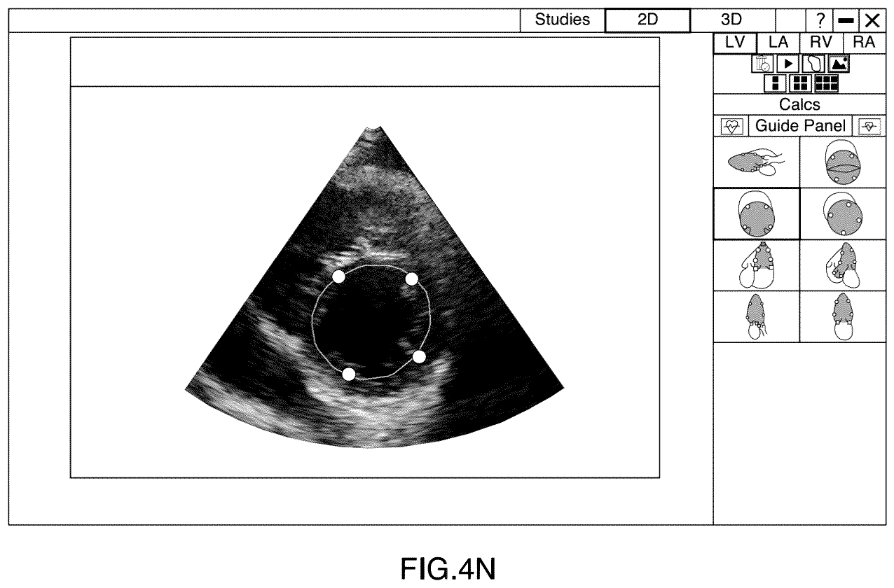

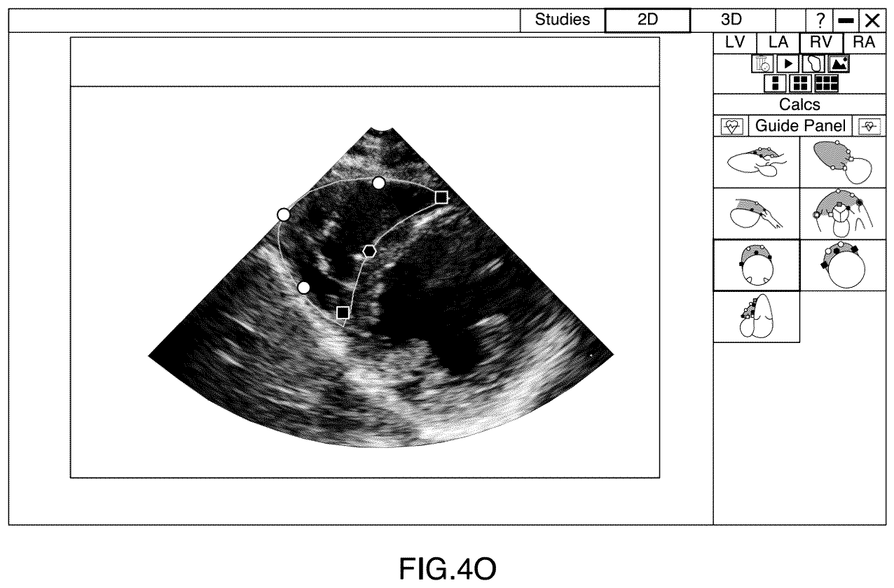

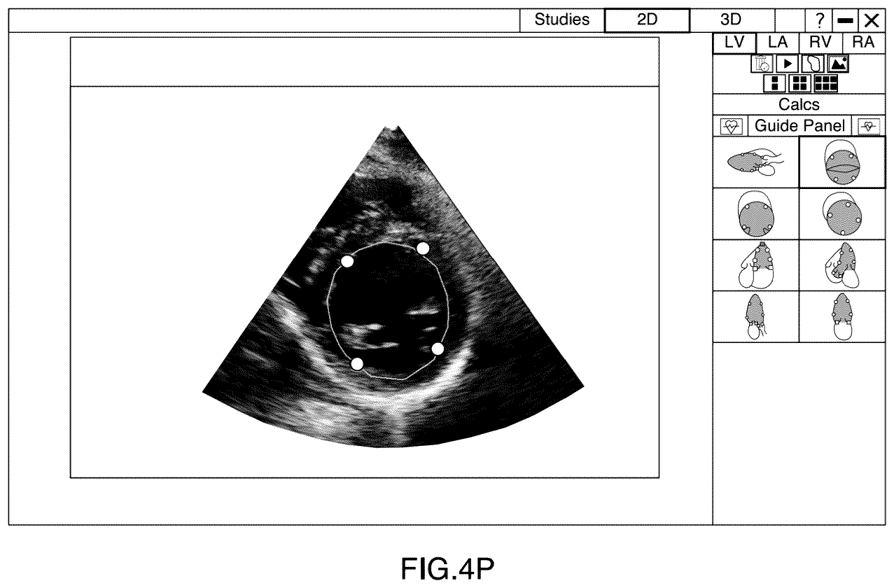

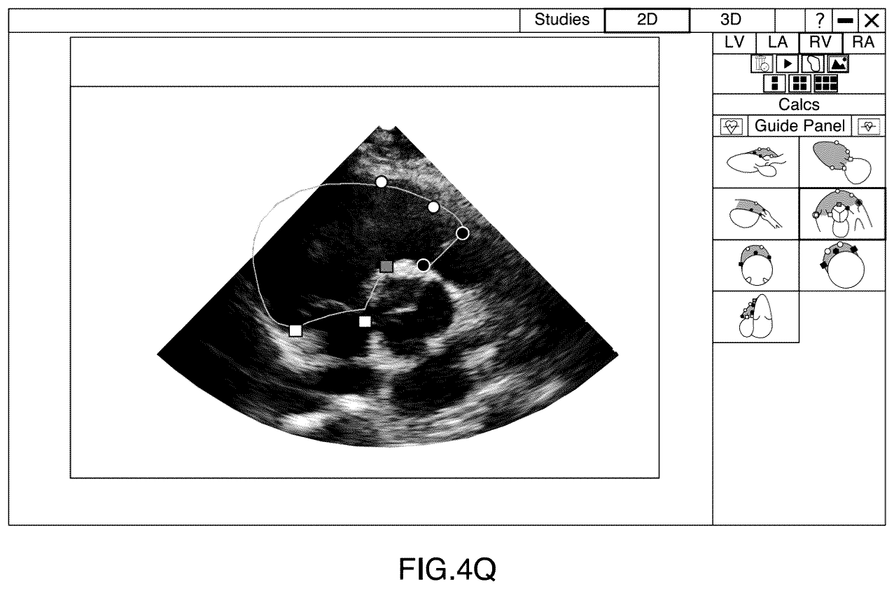

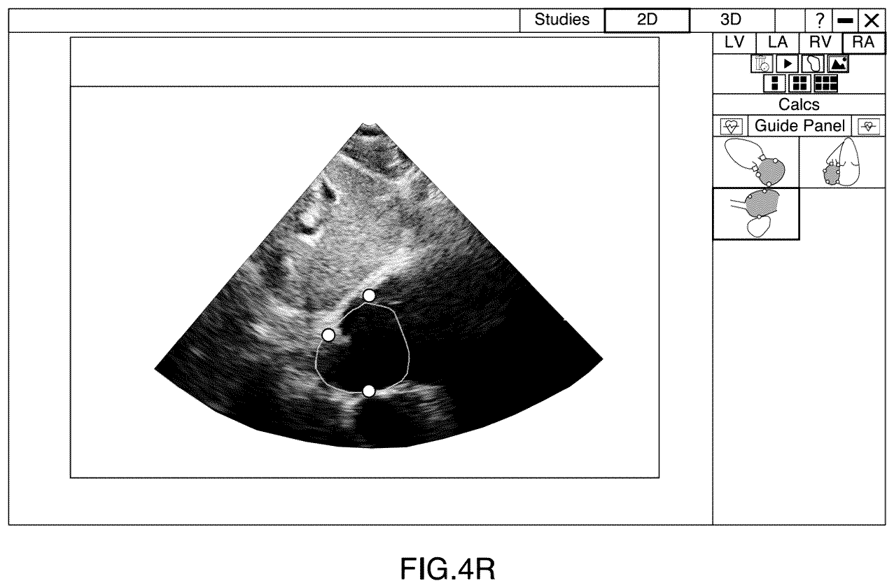

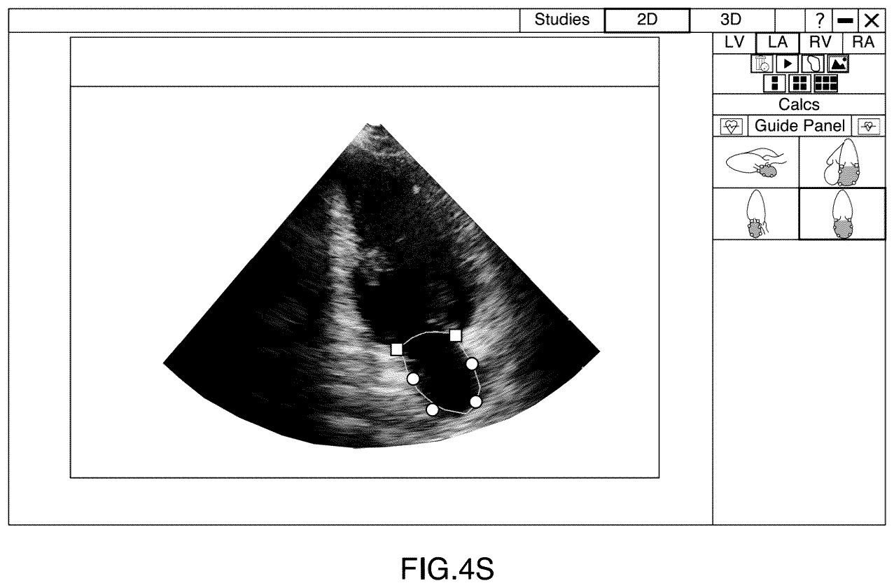

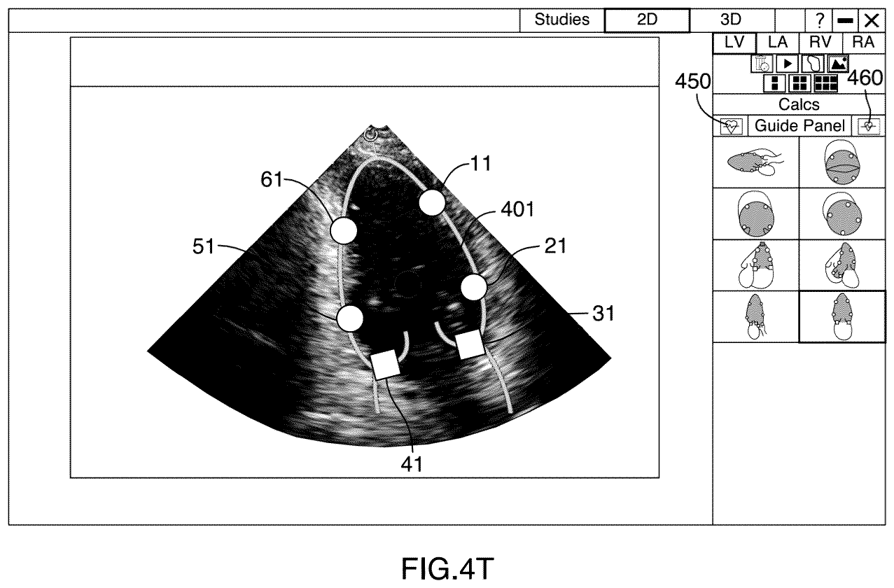

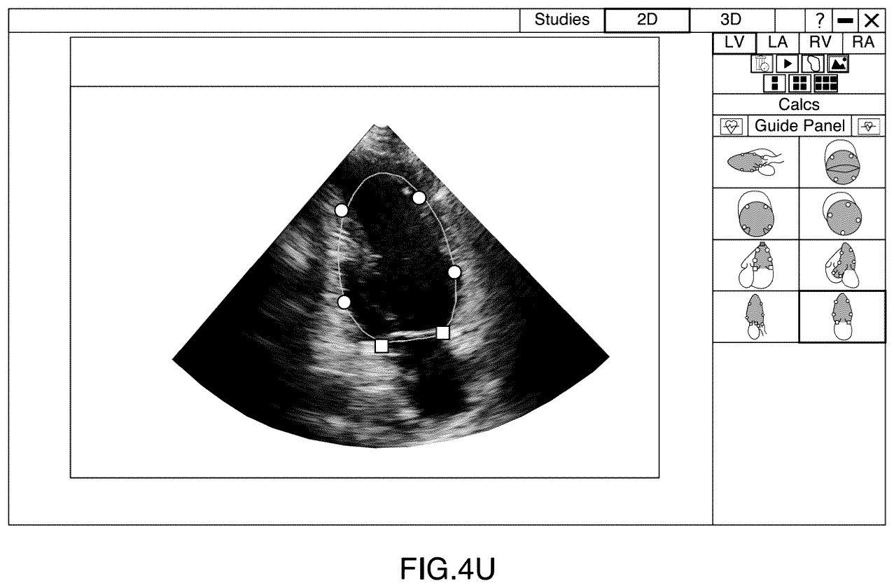

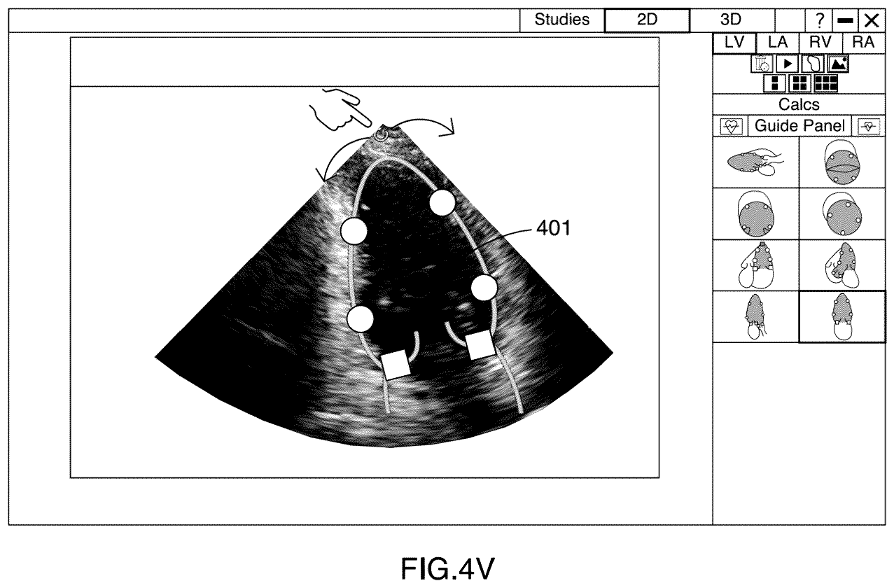

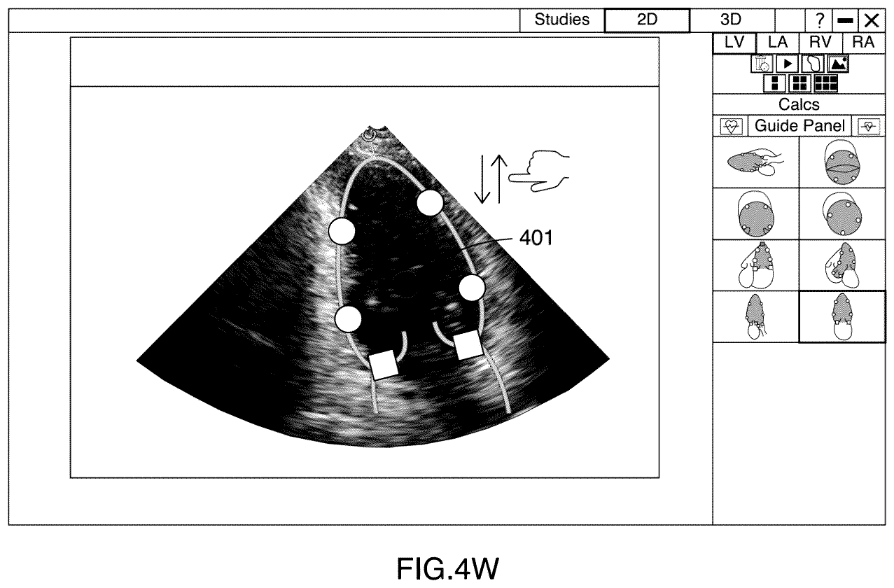

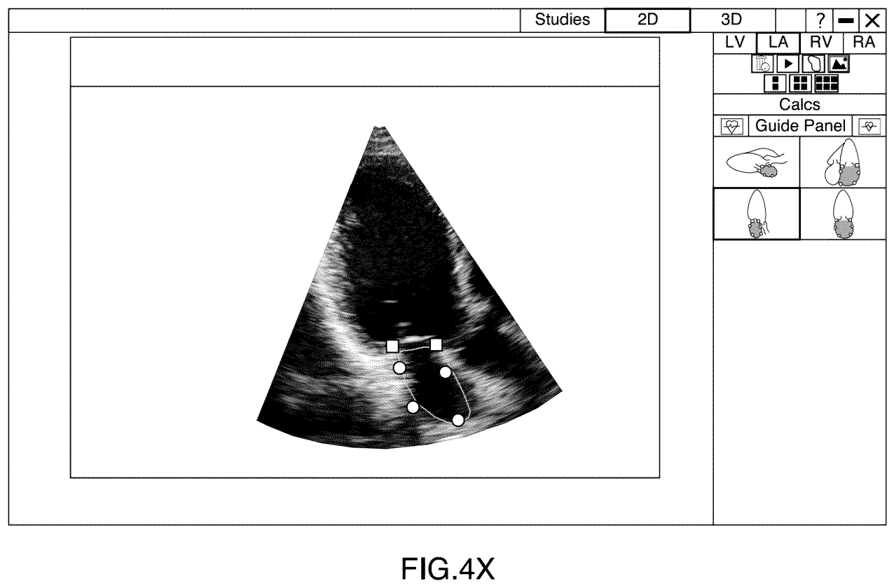

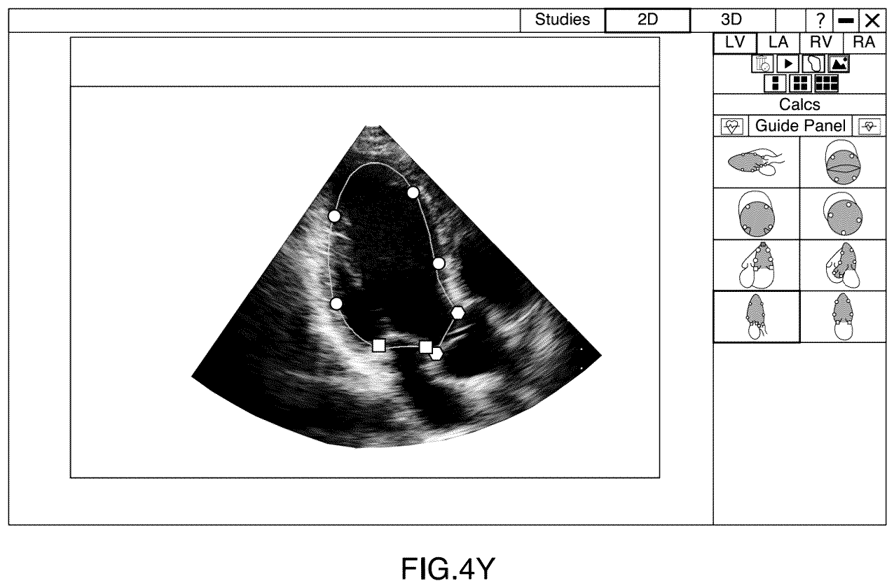

Aspects of the present invention are directed to 3D echo “slice selection”. For 3D echo slice selection, it is easier for clinicians to view and access the standard 2D images given this is the fundamentally how they are trained and, as such, are familiar with these images. The common views are parasternal and apical views that clinicians are trained to evaluate. 3D echo is relatively new and there are issues with image quality due to the lack of temporal and spatial resolution. Aspects of the present invention provide tools to assist with the slicing of the 3D model into 2D views on which points are placed and then the images may be rebuilt into a 3D model from which the cardiac measurements are derived. In a preferred embodiment, the software of the present invention can display a guide of the required guide images or views for the chosen chamber (e.g. LV, RV, LA, RA) in a main or “STUDIES” Screen (see 105 in FIG. 1A, for example) displayed to the user via the user interface (e.g. the GUI 100 in FIG. 1A). In a preferred embodiment, the software of the present invention can allow the user to choose the chamber to image in from the main or “STUDIES” Screen. As shown in FIGS. 4A to 4Y, there is provided 2D point placement (see for example points of 318B in FIG. 4A) for each of the guide images provided in FIG. 3B that are used to guide the user to tag or select representative slices.

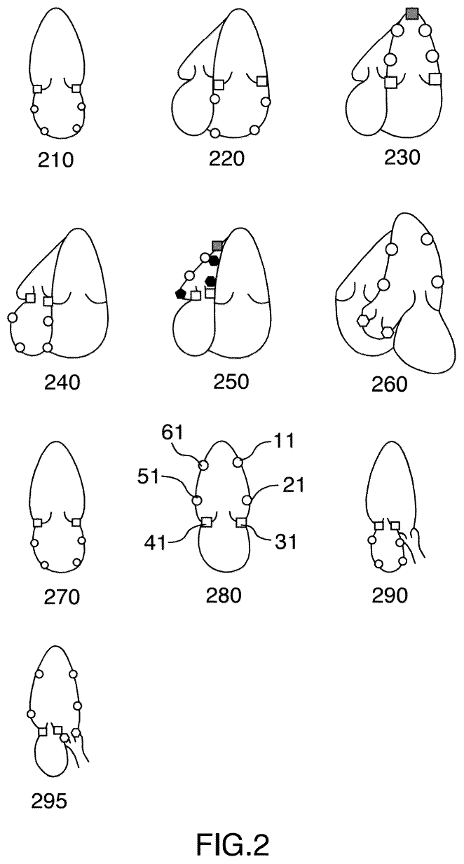

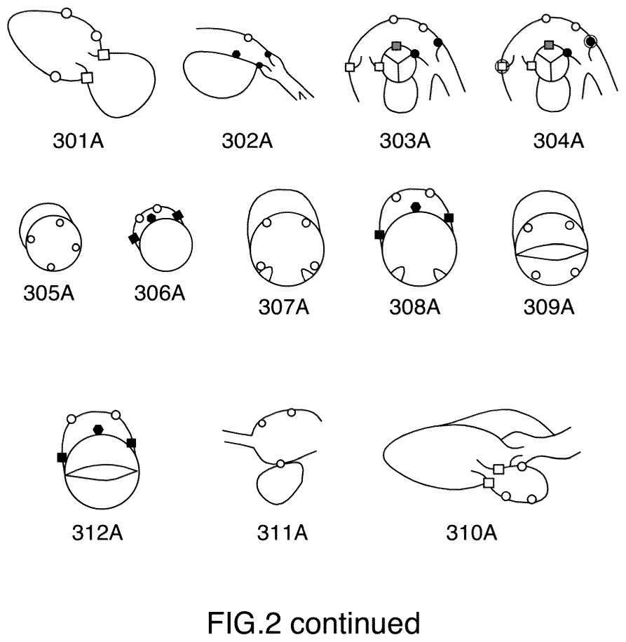

In a preferred embodiment, the slices or views are selected from the echo-based or MRI 3D datasets, which can be imported into the software of the present invention (see FIGS. 1B to 1F). The analysis then uses the KBR technology to further process the slices using the selected or applicable templates/overlays of the present invention. The present invention may then provide user interface and workflow to increase the ease with which a user can select the slices that can be used for the KBR process (see for example, FIG. 1A). This solution allows users to quickly and easily employ protocol images (see 105 in FIG. 1A), templates/overlays (see FIGS. 2, 3A and 3B) to help place the anatomical points on the distinct slices (manually or through the use of A.I.), and use tensor analysis as described in U.S. patent application No. 63/205,658, the contents of which are hereby incorporated by reference. Different companies such as TomTEc, Siemens, Philips and GE each have products that determine volumes in different ways. MRI measurement solutions are AI driven (Circle Imaging, Calgary) but do not provide ease of use interfaces and workflows which can be used across the board for all moieties, but instead provide unique interfaces and workflows to the specific modality (e.g. commercial systems) and the software version used therein.

In a preferred embodiment, the system, method and/or computer readable medium of the present invention relies on any modality adapted to obtain cardiac image data and import the image data to allow “slices” along separate intersecting planes for mapping to particular templates (see FIGS. 1B to 1F). Persons having ordinary skill in the art may appreciate that typical modalities for obtaining cardiac image data in the prior art include 2D and 3D echo as well as MRI. The first step is to convert the image data into voxel size and voxel grid dimensions where a voxel is a 3D pixel.

In a preferred embodiment, as provided in FIGS. 1A, 4A to 4Y and 5A to 5V, there is provided a GUI 100 to the user which in turn has a dashboard that displays one or more of the medical image data (e.g. 3D echo), the slices, the templates and applicable task bars. As can be seen from the dashboard, the user may be provided with a display on the screen of one or more of the following: (a) the image data, including 3D echo data and 2D representative slices (see 101, 102, 103), (b) representative model of the 2D slices based on one of three positional and rotation axis (see 104) to provide a 3D representation of the structure under examination (e.g. a “3D Cube”); and (c) the protocol images based on anatomical structures (see 105). The use of the protocol images permits a user to identify and determine what 2D slice the user may want to select using the position and rotation along an image plane (e.g. XY, ZY or XZ) as well as the voxel grid information. A 3D matrix transform is created from the information to calculate the origin, the applicable plane and direction, as well as the image data for that slice. The image data can be obtained via interpolation, or if applicable, by querying the 3D echo data using the plane information. As a user moves a selected plane in the 3D Cube view window, the corresponding 2D plane image adjust to provide the applicable image. When a user needs to import a 3D Echo study into the VMS+ software of the present invention, the 3D echocardiogram needs to be sliced into 2D planes, planes that match a standard 2D echocardiogram study (see FIGS. 1B to 1F).

The current process is to work with 2D images extracted from the 3D however this is not very intuitive (e.g. see FIG. 1B). In the process of the present invention, the user will be able to interact with a 3D model to identify and extract the scan planes.

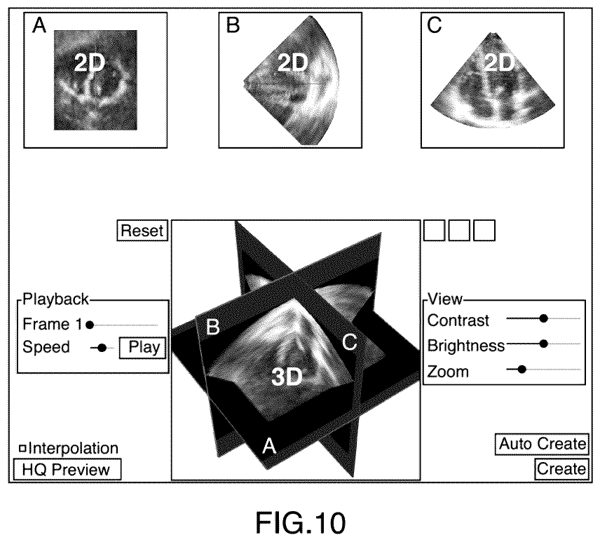

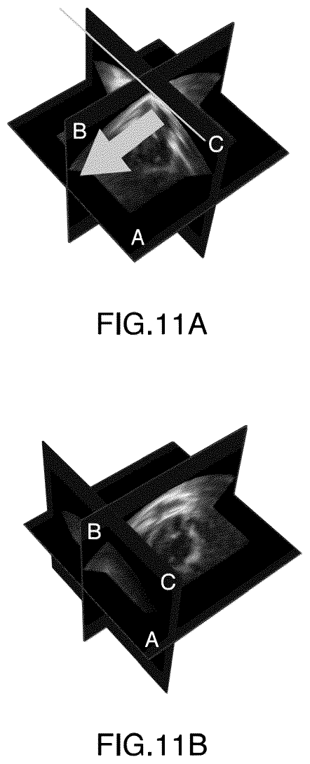

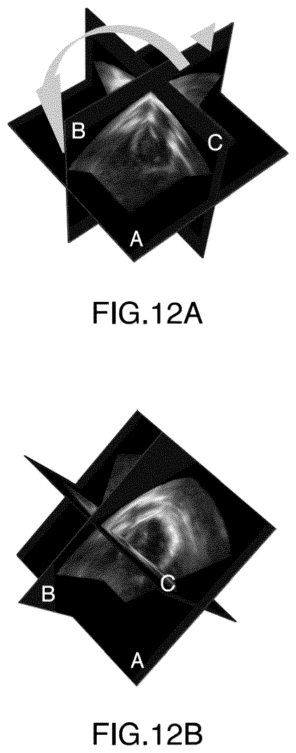

The VMS+ software of the present invention may have the 2D and 3D components (see FIG. 1) in one screen and the user can interact with 2D, 3D or both items to extract the views of choice. As shown in FIG. 1A and FIG. 10, the three scan planes on the top row (which, in a preferred embodiment are color coded green, red and blue) have the same corresponding orthogonal planes in the 3D Cube (see, for example, bottom middle image of FIG. 10). The 3D Cube contains three scans placed orthogonally with the user moving and rotating the planes within the 3D Cube-essentially, the user is interacting in real-time with the 3D Cube. In a preferred embodiment, the 3D Cube can be spun around so that all sides of the cube can come into view. See also, for example, FIGS. 11 and 12. The user can play the captured cine clip (ED to ES) in the 3D Cube as they interact with it. As the frames are moved manually by the user, the cube is updated based on position of user. In playback mode, the cube displays to the user an automatic stepping through the frames and updating the frames at the rate of playback.

Moving the planes requires touching a long edge of a plane and dragging it to the desired location (see FIG. 11A and 11B).

Rotating a plane requires touching a corner edge of a plane and dragging to the desired orientation (See FIGS. 12A and 12B).

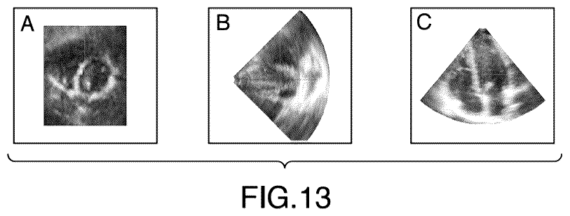

This allows the user to easily see where the scan planes are relative to each other-instead of the current process which the user must mentally visualize where the scan planes are relative to each other. This aspect of the present invention may allow more users to easily extract the planes required to run reconstructions. In the end, the user can visualize how the 3 scan planes relate to each other and how to extract the planes (see FIG. 13).

In a preferred embodiment, GUI 100 may also allow the user to save specific slices in a thumbnails panel (see 106 in FIG. 1) as well as select and edit any saved slices from this thumbnail panel. In FIG. 1, no slices have yet been saved. Saved slices may show up in the thumbnail panel and the scans in the panel can be selected. Once a slice has been selected the user can click a remove button to remove that saved scan, if so desired.

As shown in FIG. 1, the 3D Cube view may be displayed as a three-dimensional model. As used herein, the term “three-dimensional model” refers to a rendered graphic that appears to have extension along one or more planes, preferable 3 planes (XY, YZ, and XZ). Preferably, the planes of the model are movable and the entire model is rotatable about one or more of the three axes (e.g., the X, Y, and/or Z axes) for viewing the model from different angles, and most preferably about all three axes to position the three-dimensional model in any orientation (i.e., the model preferably has three rotation degrees of freedom). To display a model, the user selects images from the three axes in the portion of the display showing the 2D image data (see 101, 102, 103). The set of 2D images selected by the user are then represented in the three-dimensional model (e.g. 3D Cube 104) for the user to observe.

In a preferred embodiment of the present invention, when a user wants to import a 3D echo study into the software of the present invention (e.g. the VMS+ software), the 3D echocardiogram can be sliced into 2D planes, planes that match a standard 2D echocardiogram study. In a preferred embodiment, the process can provide the ability to work with 2D images extracted from the 3D data set. However this is not intuitive to the user. Through the process of the present invention, the user will be able to interact through the user interface (e.g. 100) with a 3D model to extract the scan planes or slices that are desired. In a preferred embodiment, the 3D Cube contains three scans or slices of different planes placed orthogonally with the user moving and rotating the planes within the 3D cube. This may allow the user to easily see where the scan planes are relative to each other—instead of the current process which the user has to mentally visualize where the scan planes are relative to each other. As a result of the embodiments of the present invention, users can more easily extract the planes required to run reconstructions.

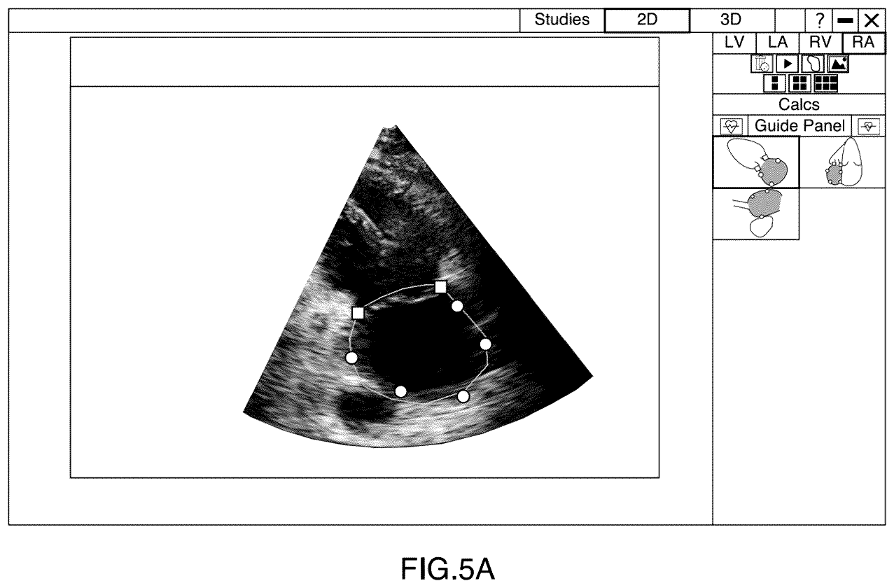

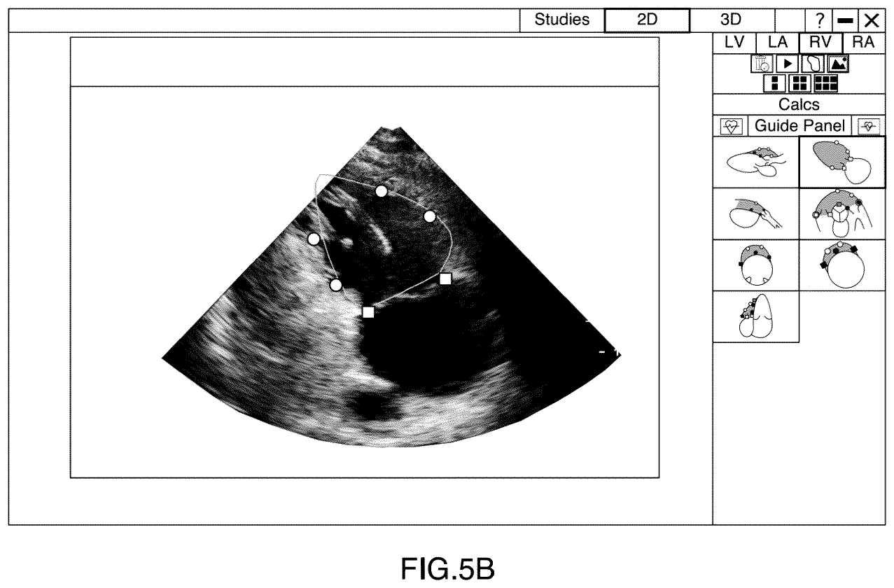

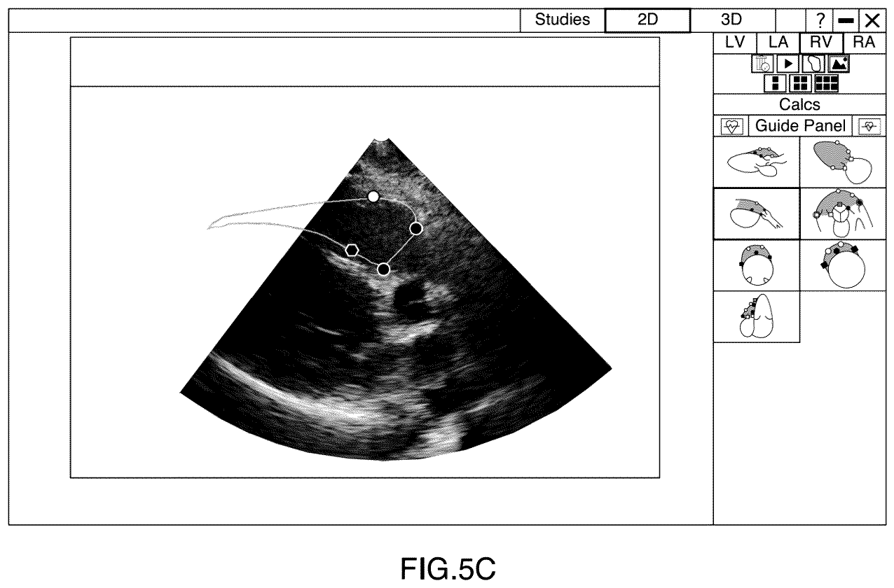

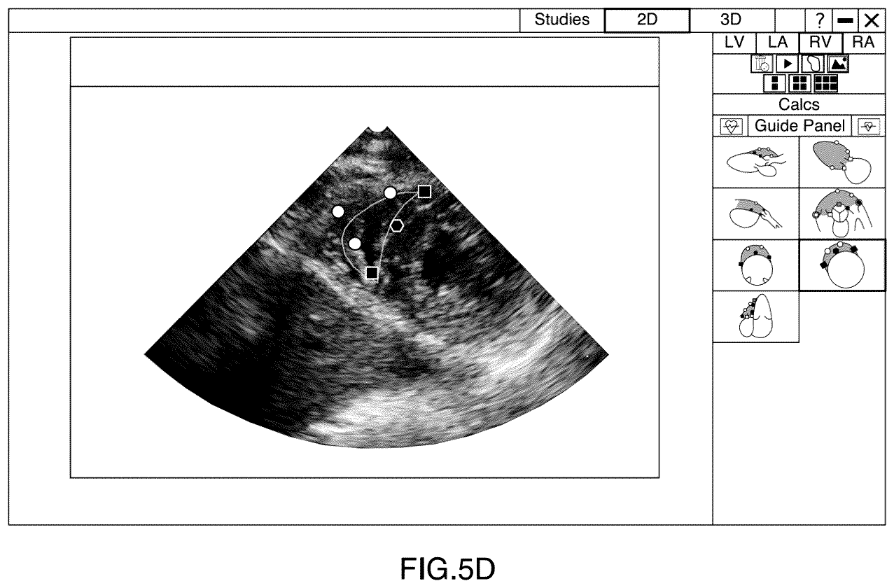

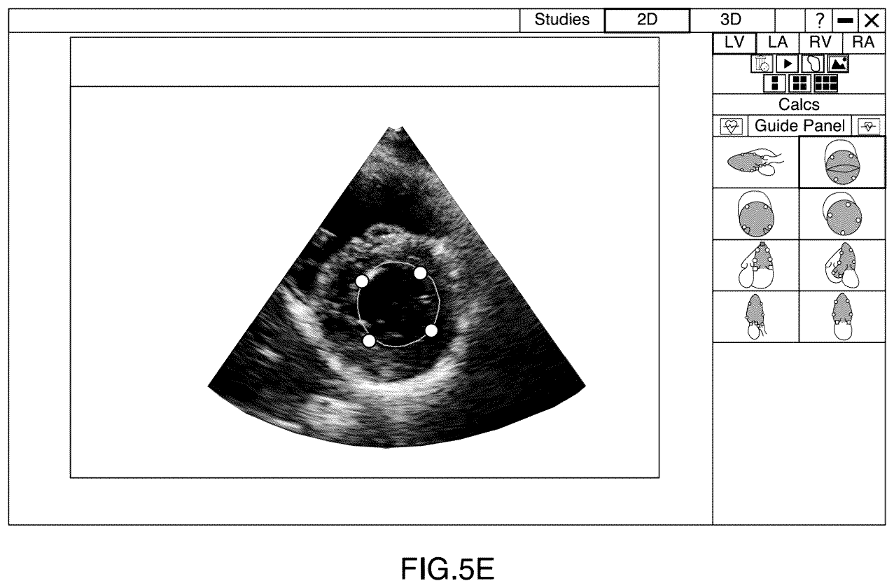

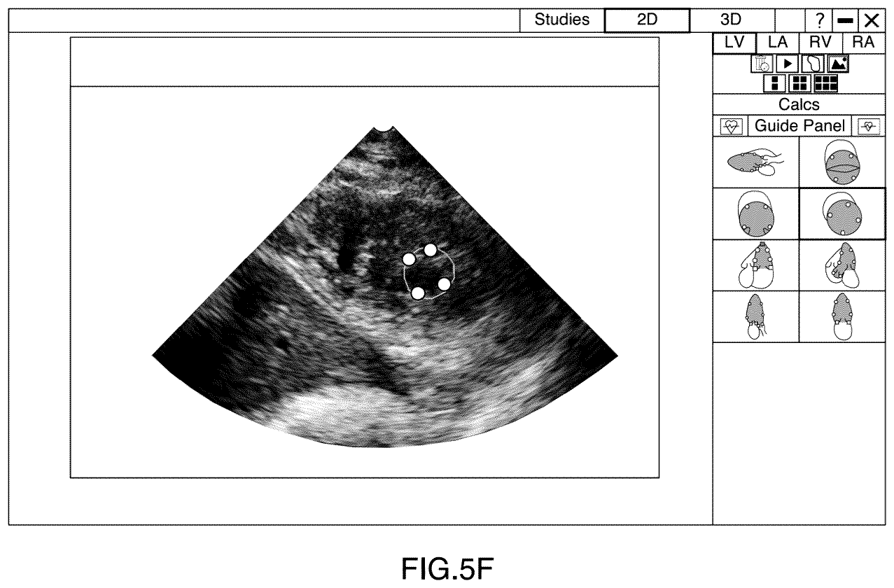

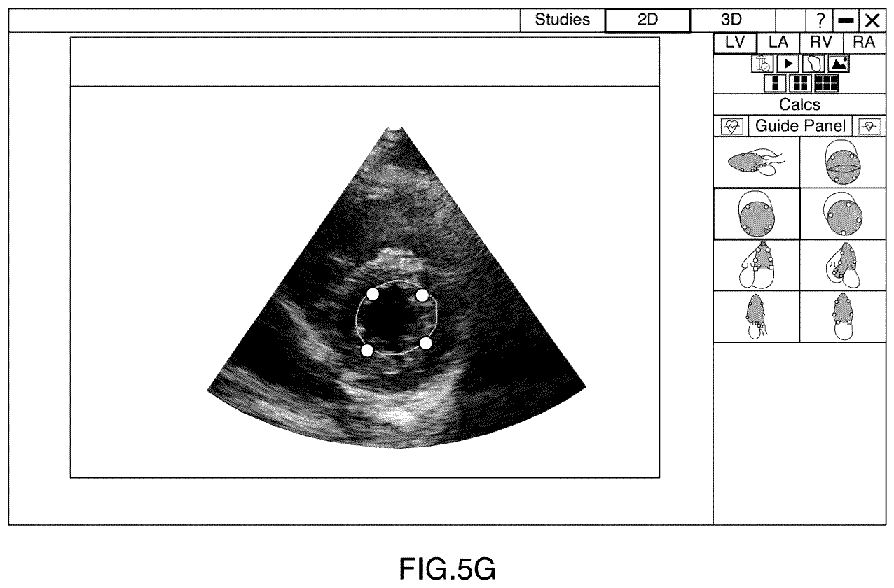

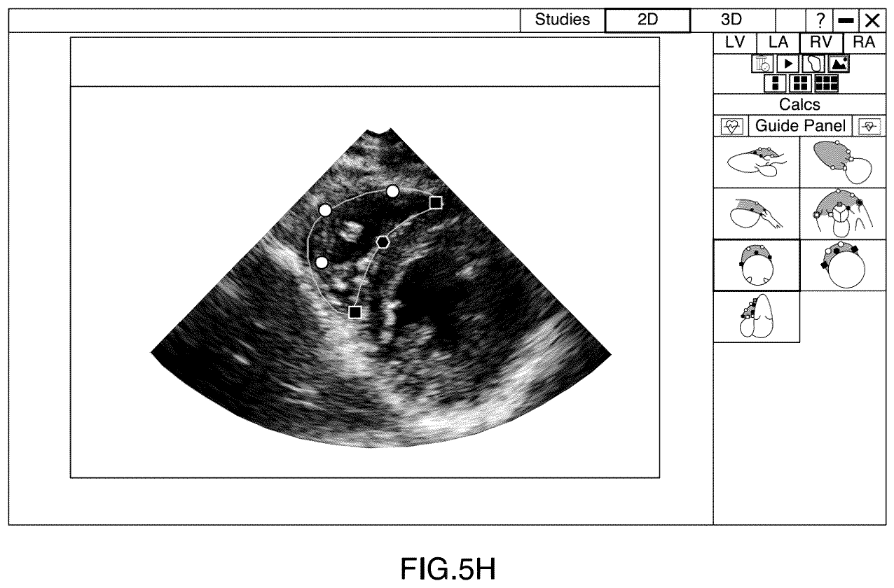

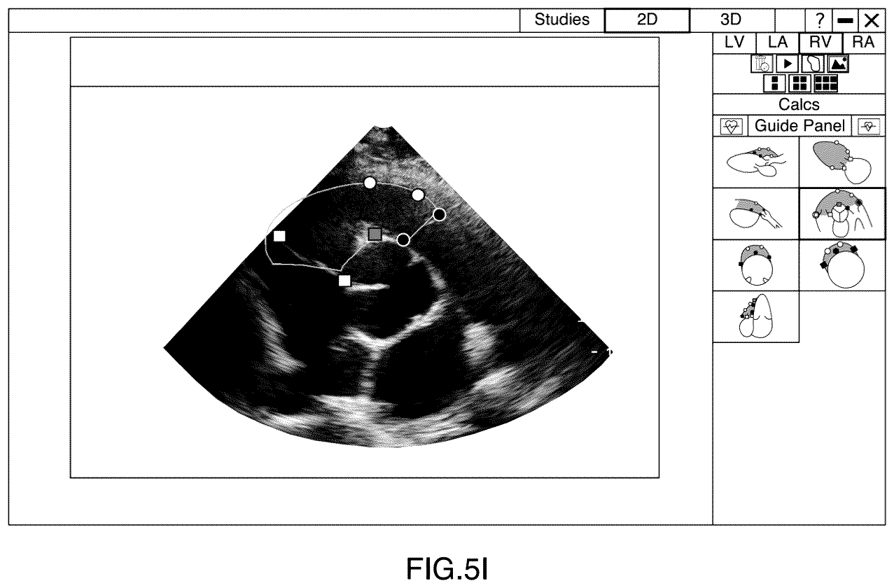

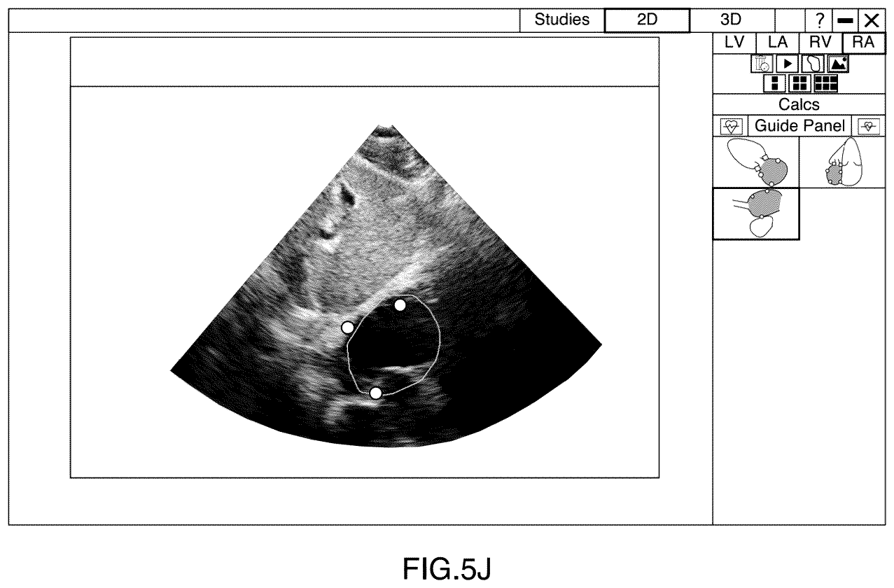

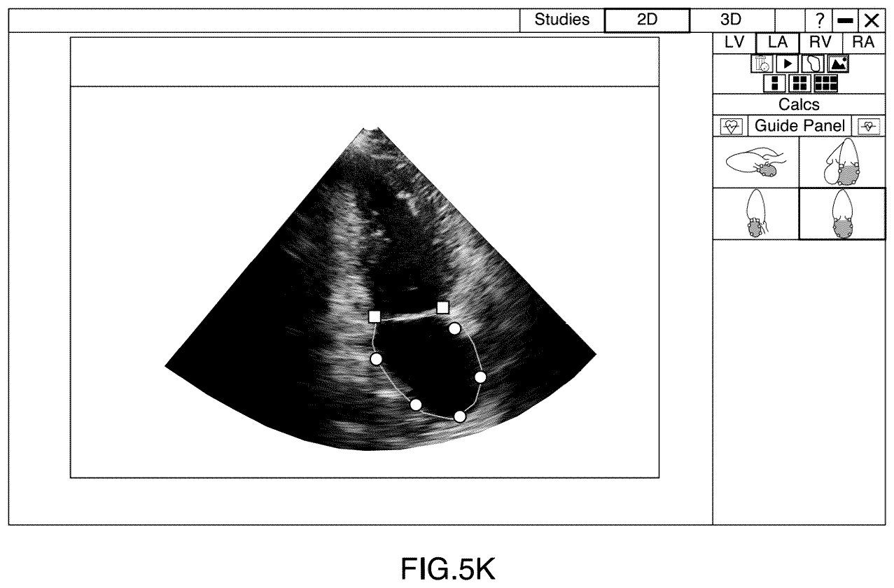

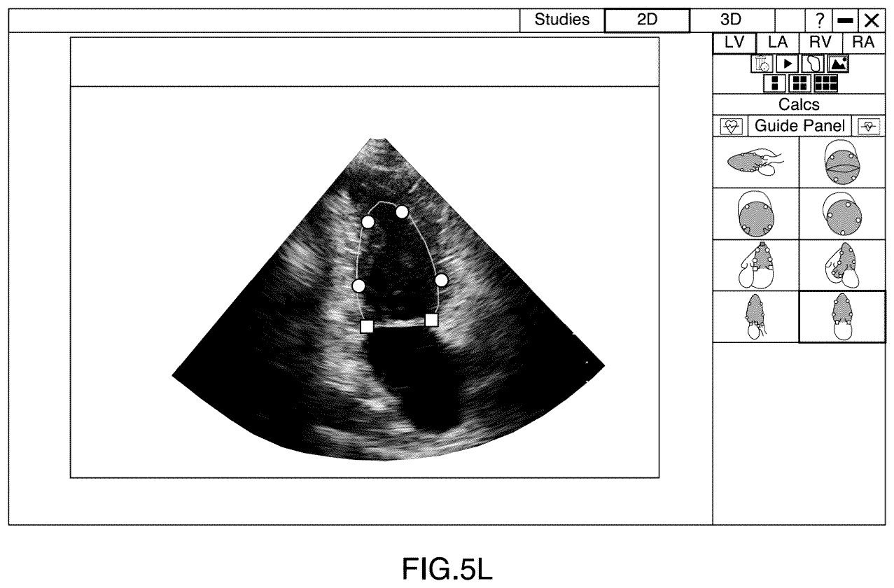

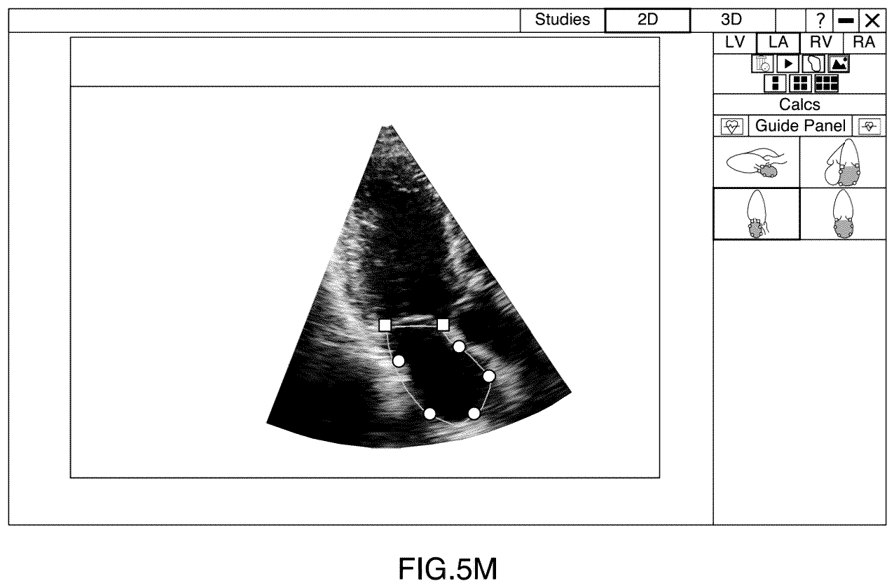

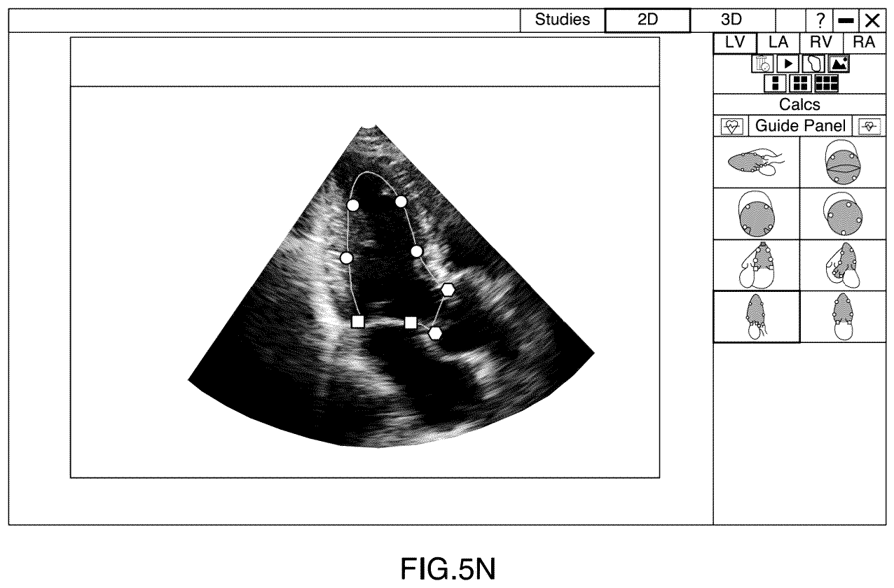

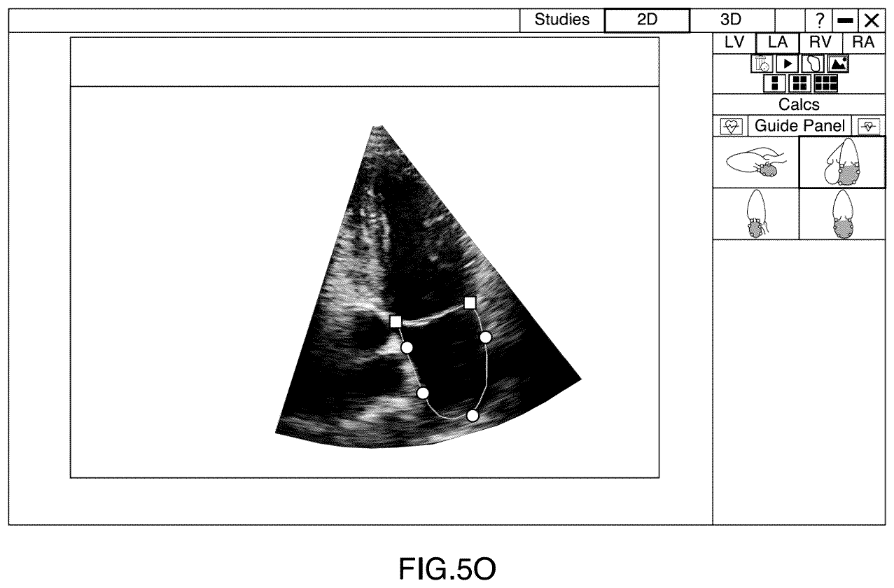

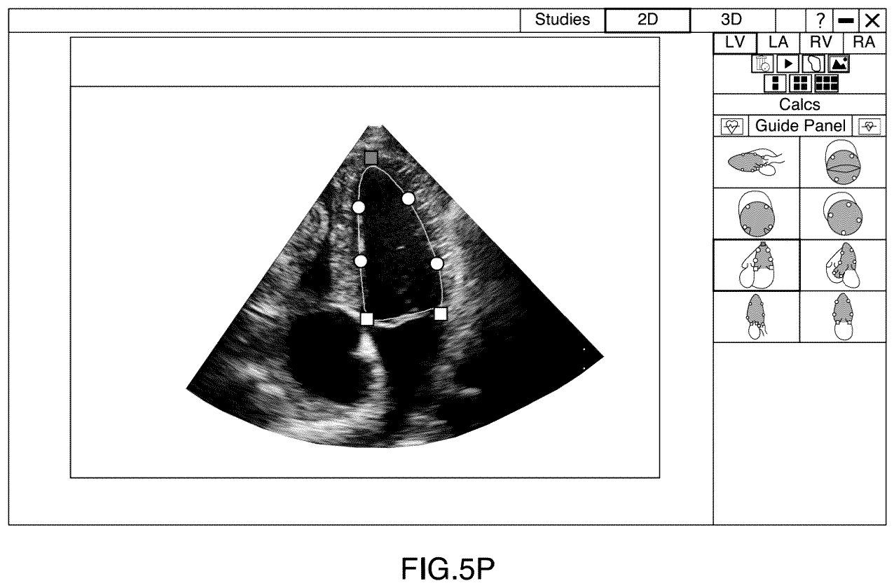

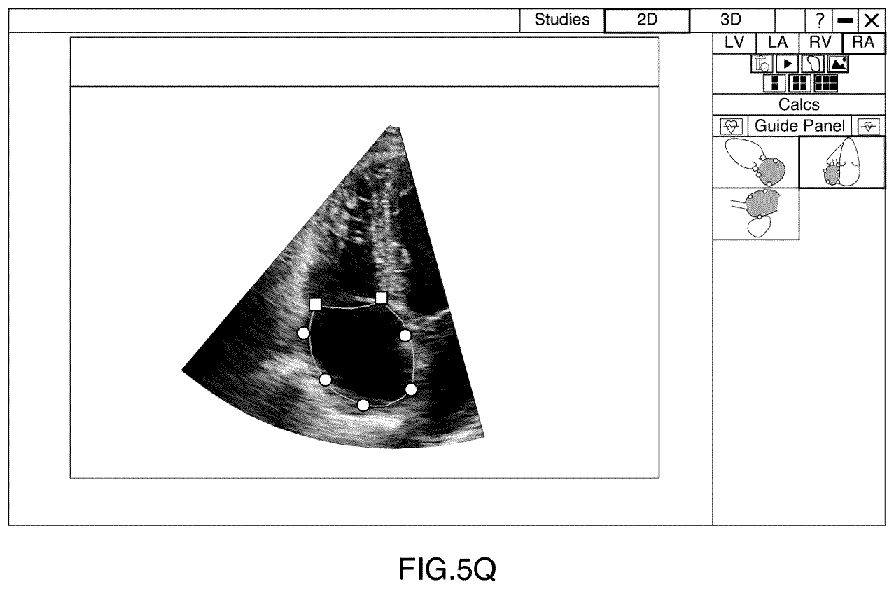

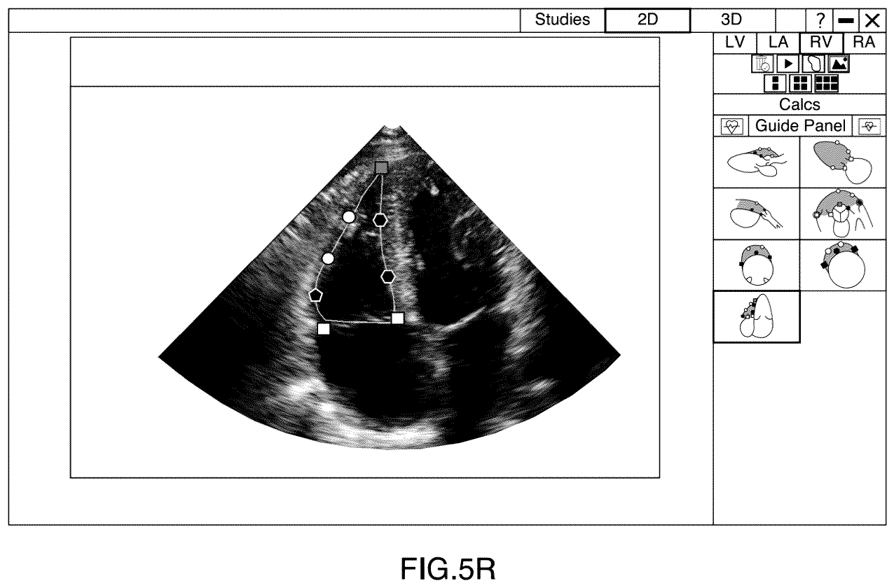

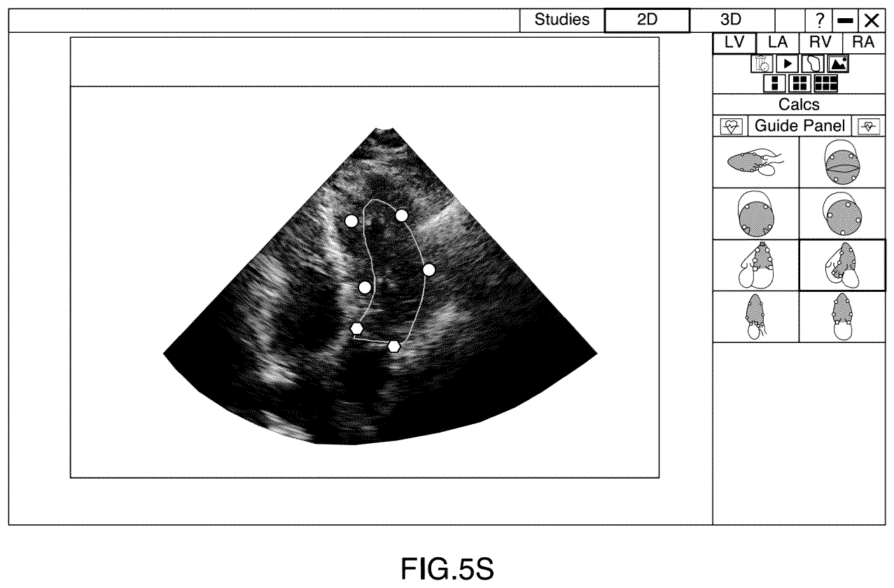

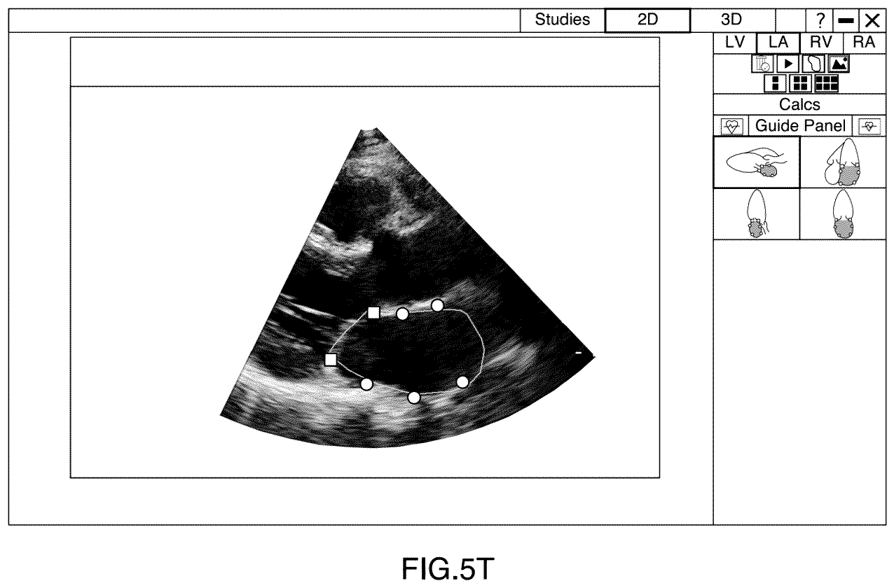

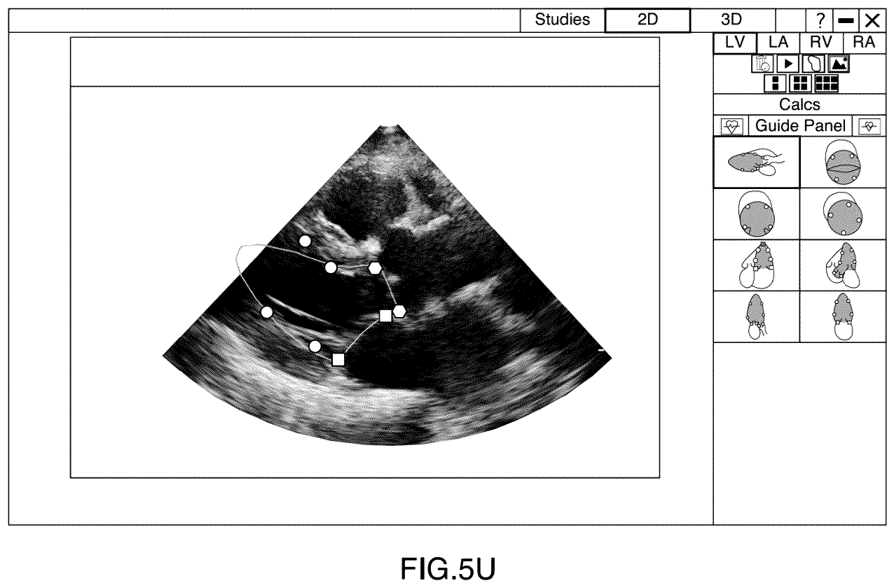

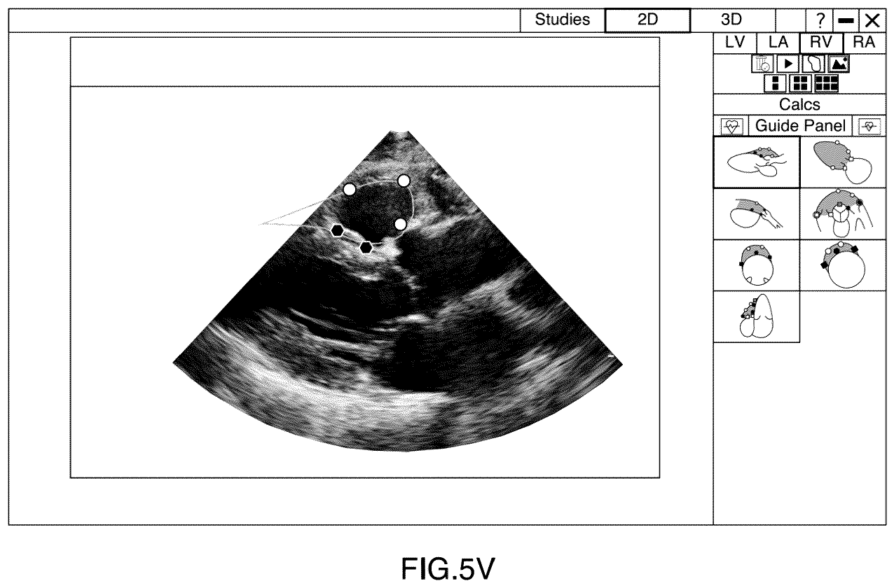

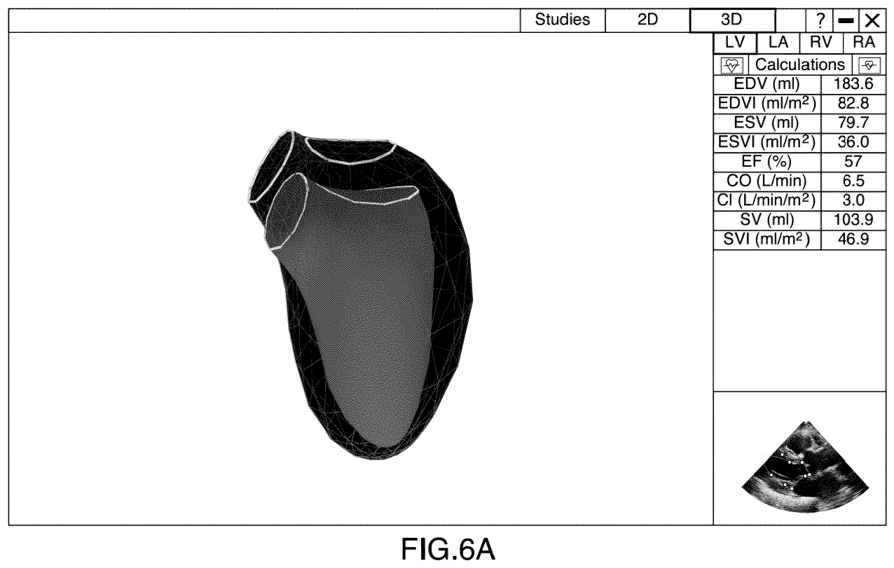

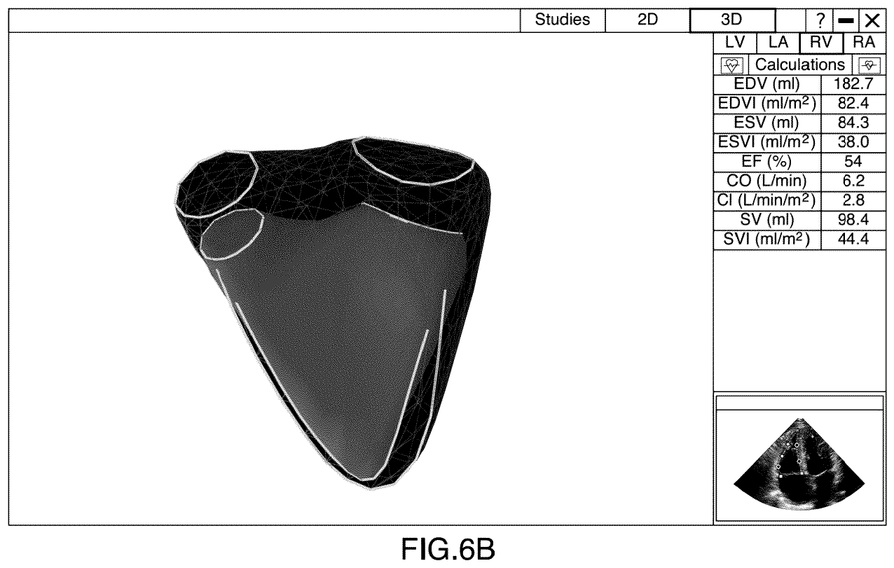

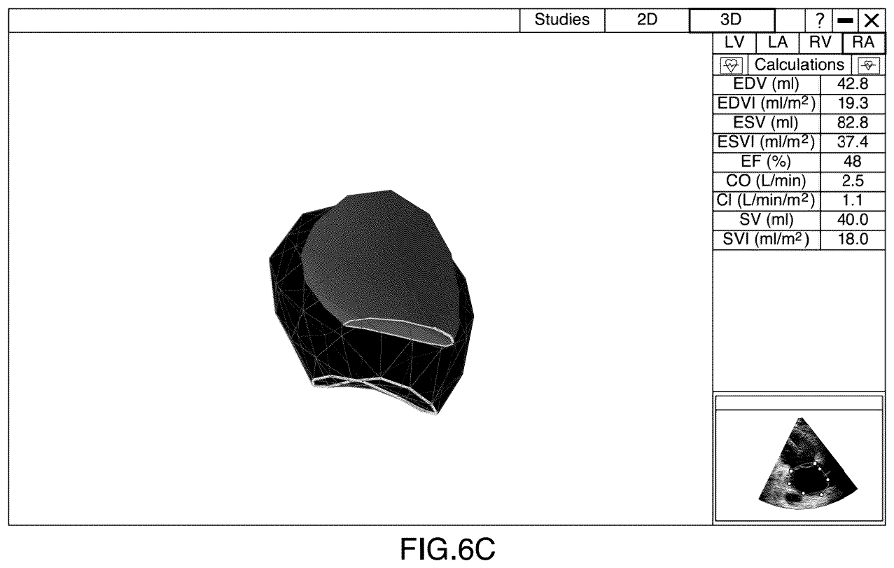

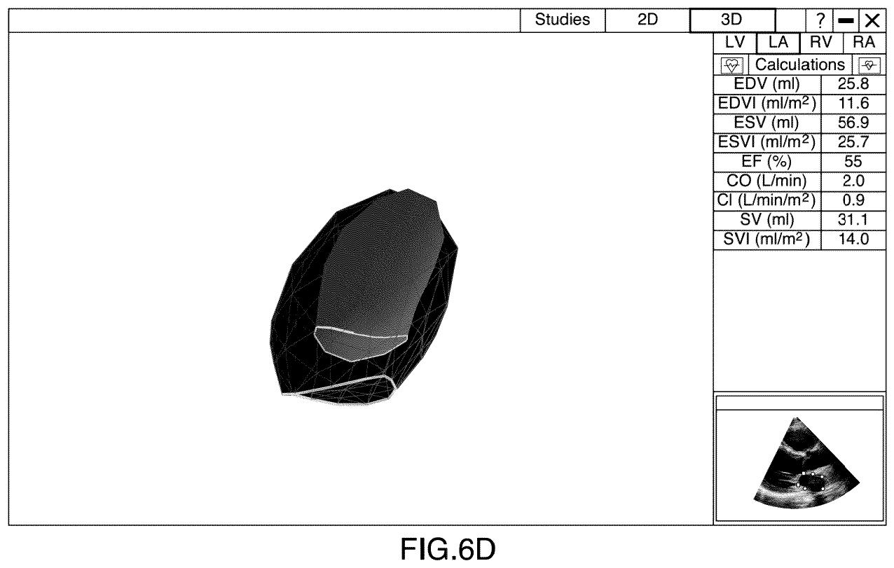

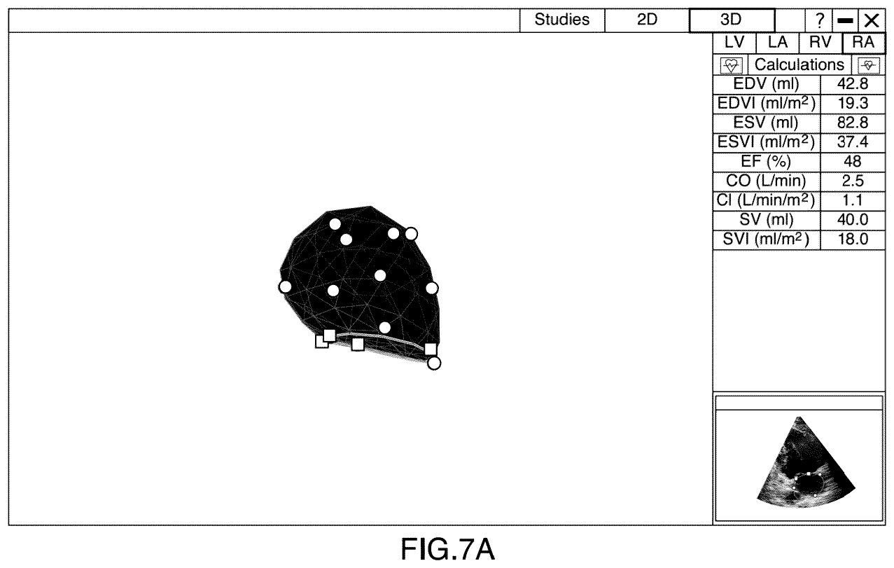

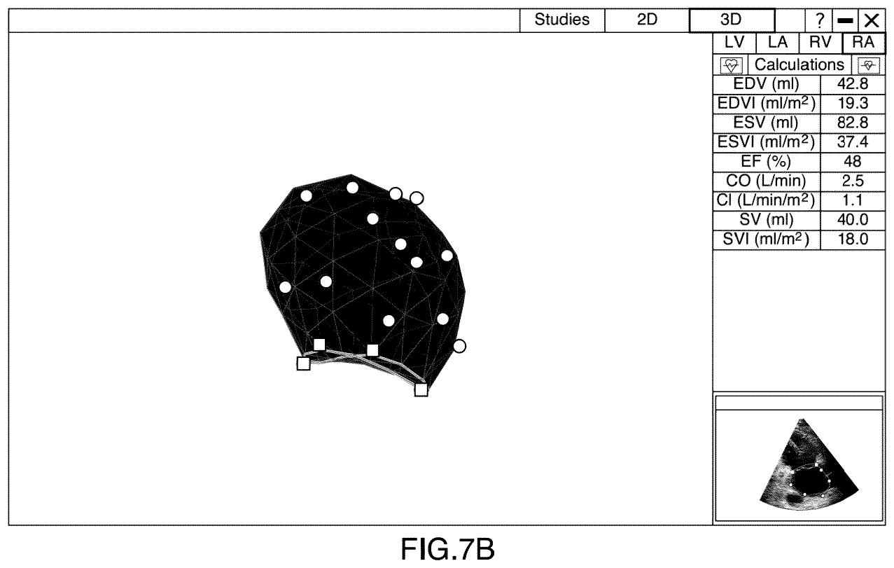

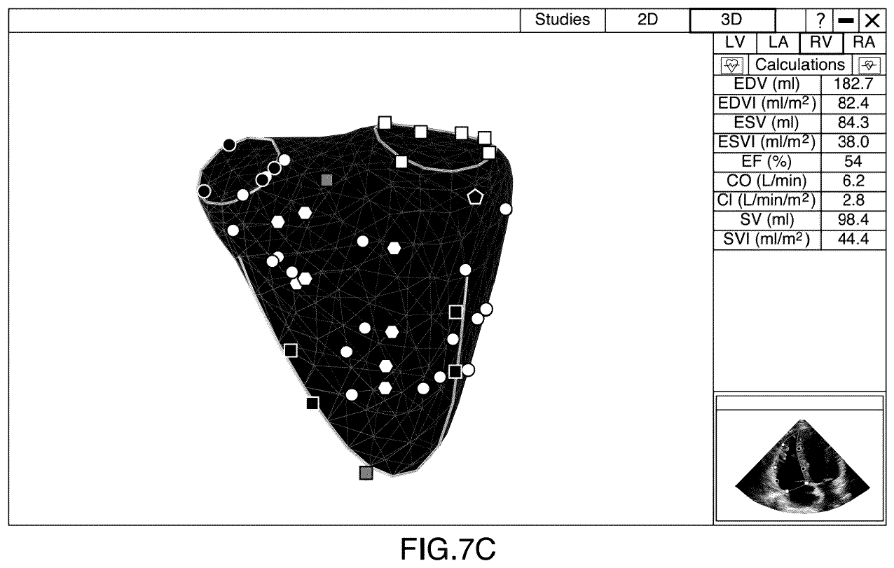

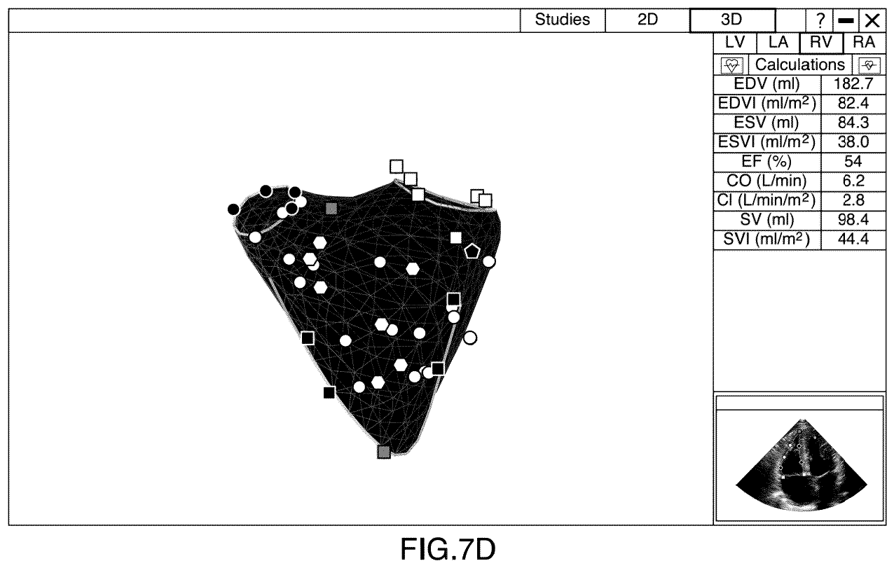

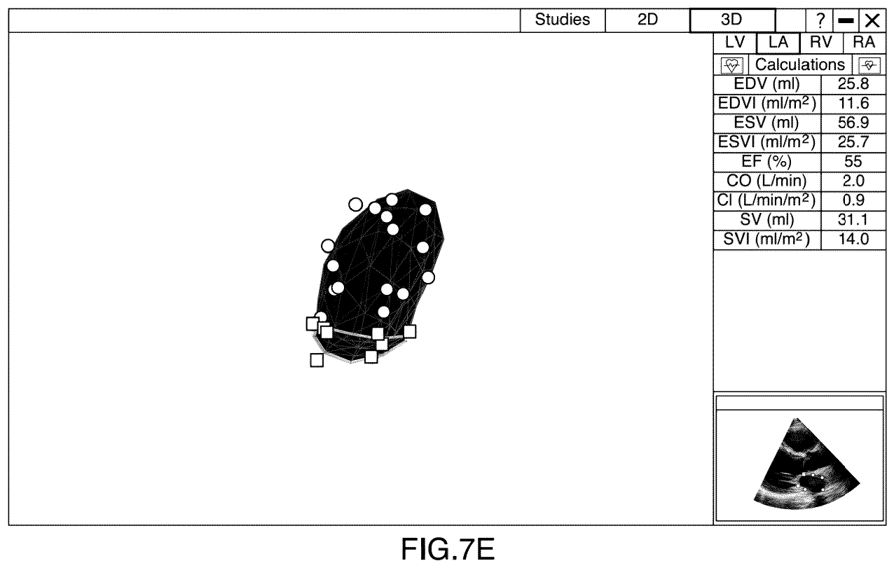

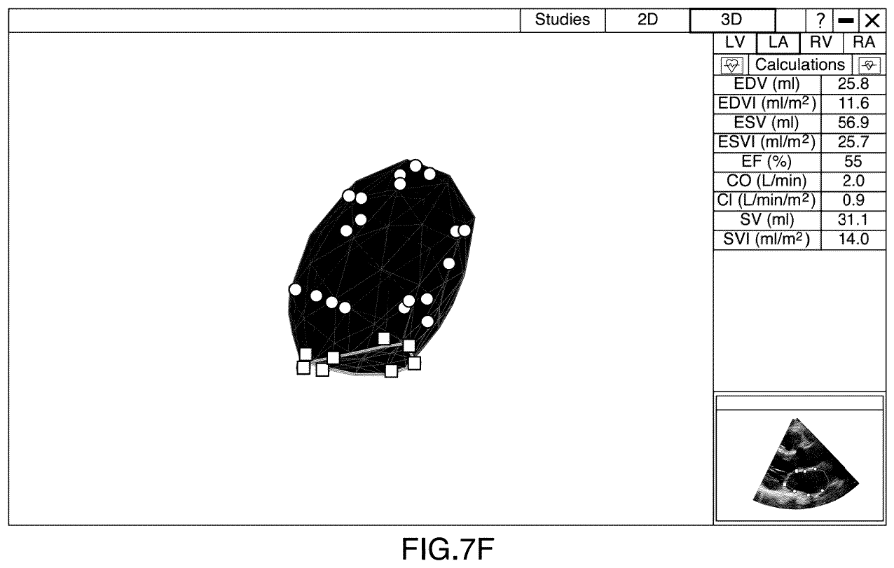

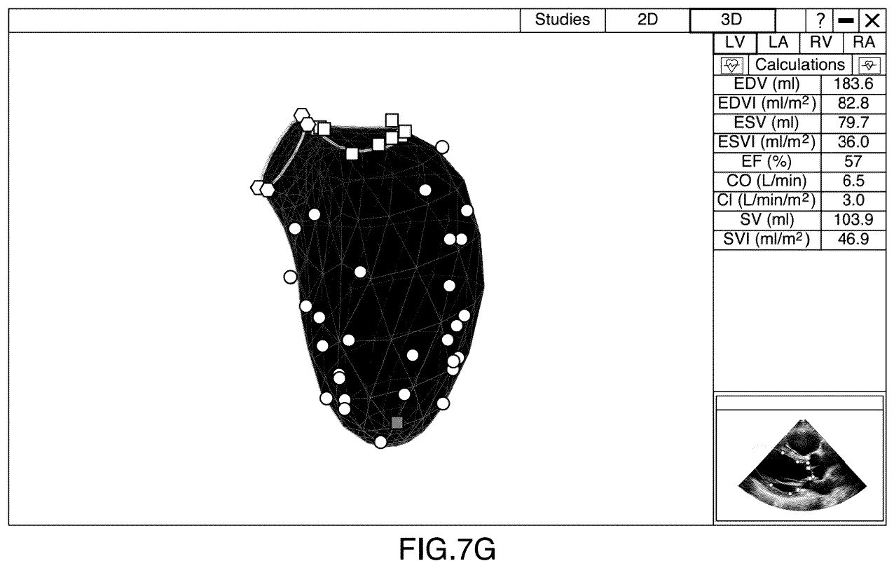

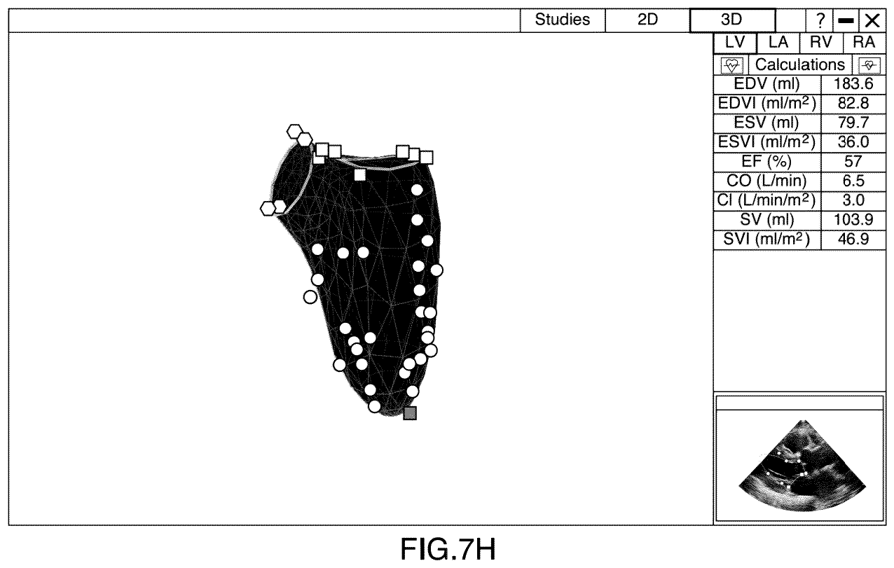

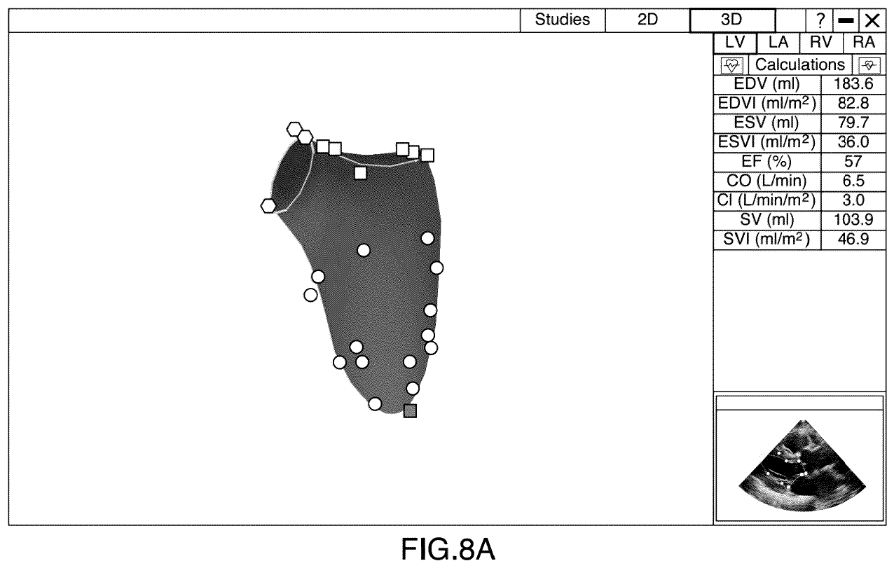

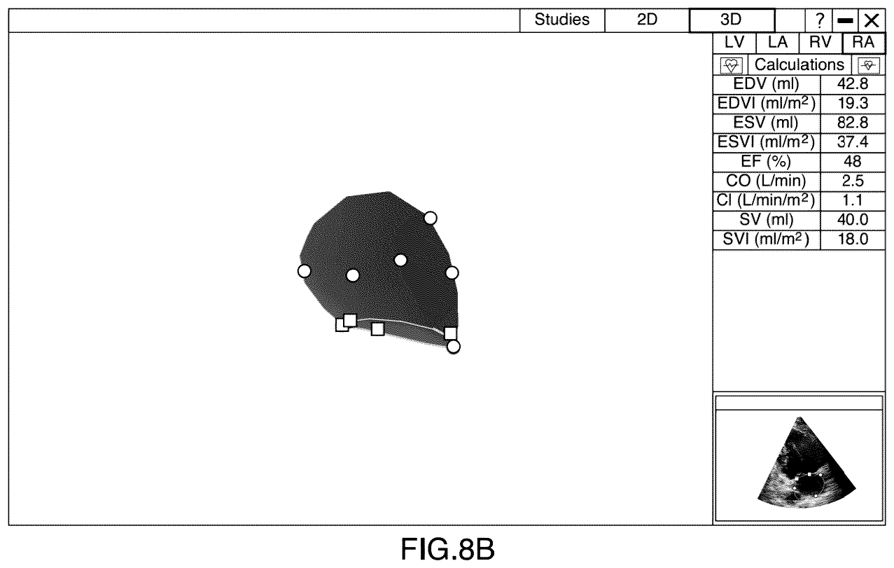

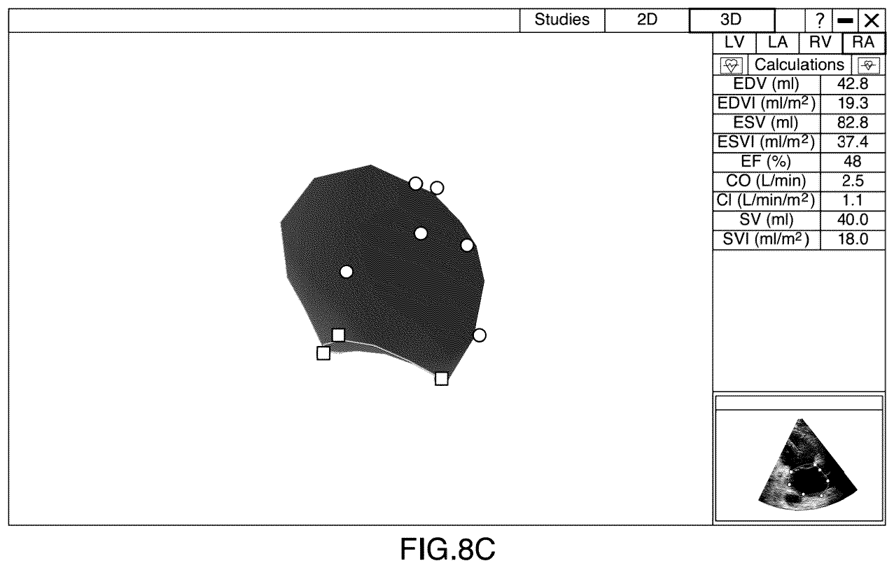

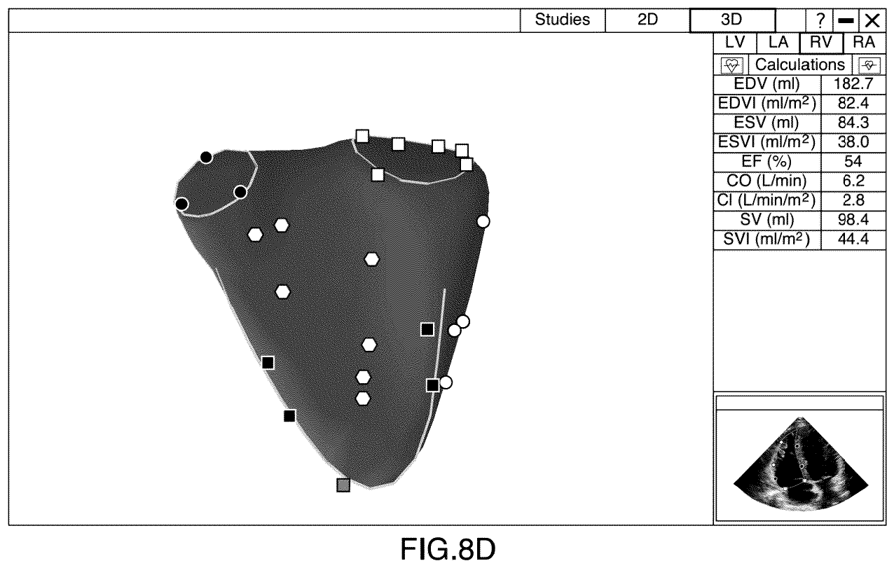

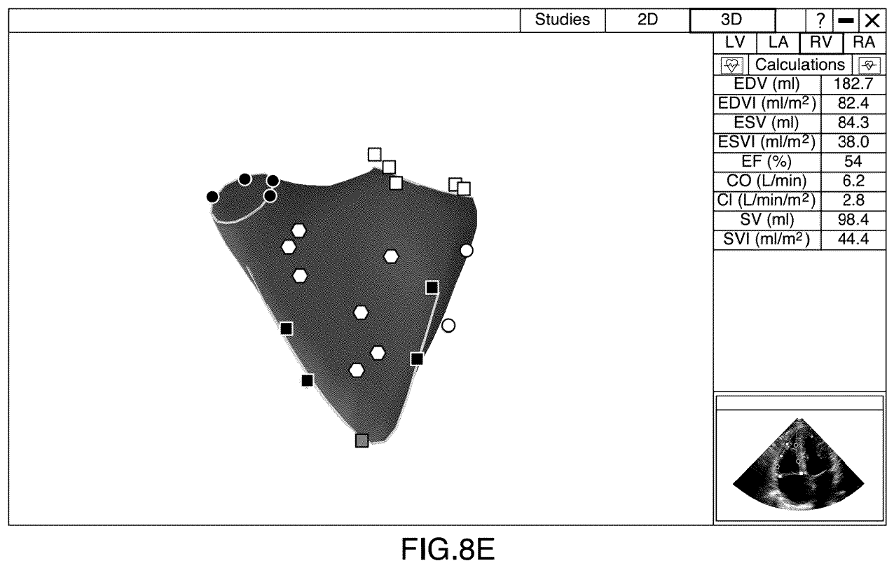

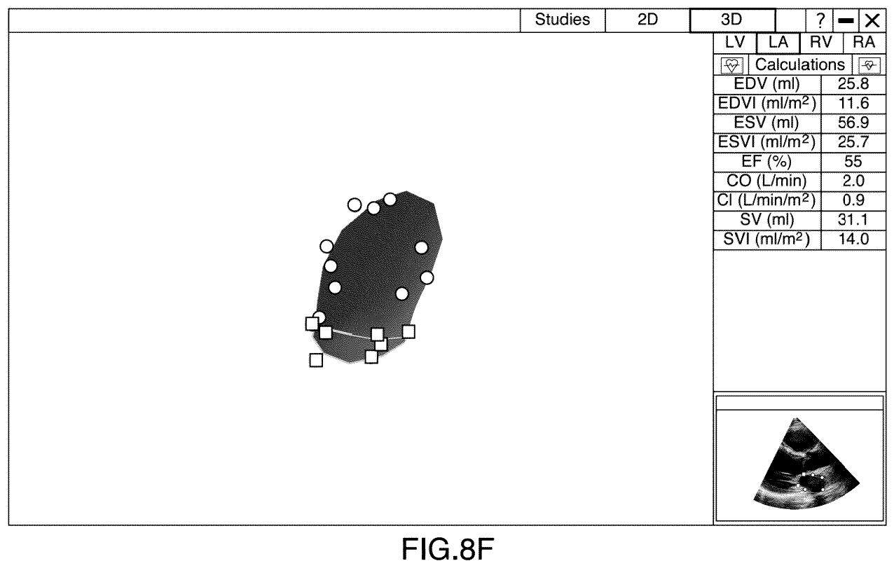

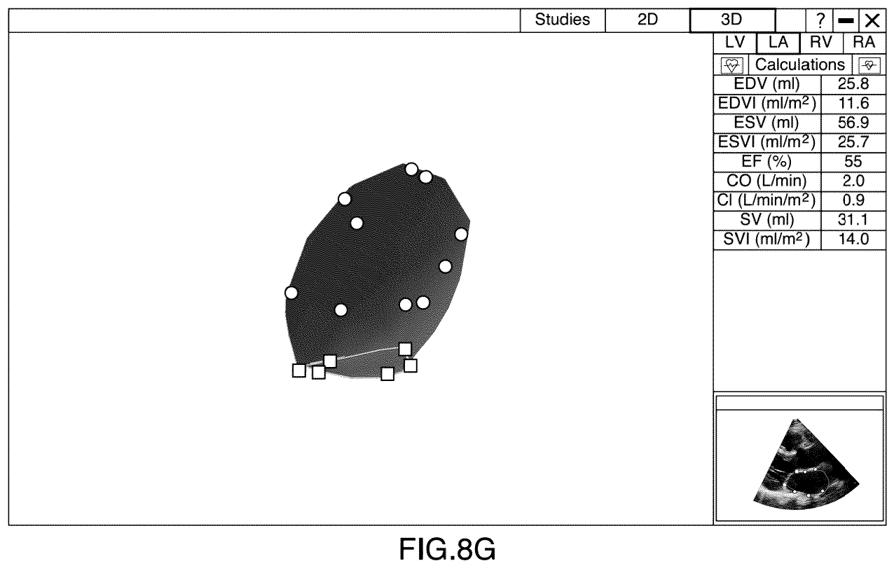

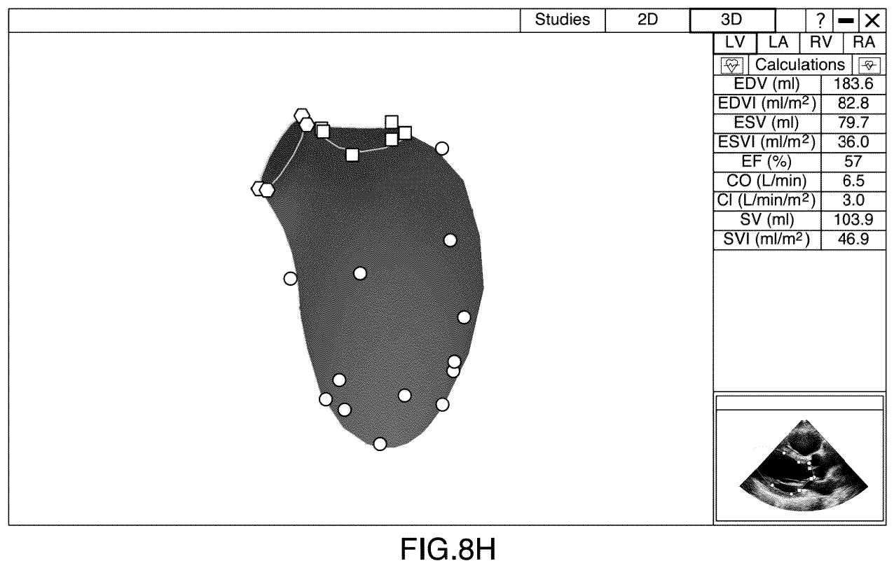

Another aspect of the present invention is 2D “point” placement within the 2D slices selected by the user (see, for example FIGS. 4 and 5). A plurality of 3D points (see overlay/template/protocol image 313A and 301B; points 10, 20, 30, 40, 50, 60, 70 and 80), corresponding to anatomic structures or landmarks of the heart on the surface of the heart can be identified by the user or through A.I. algorithms in the 2D slices. This can be achieved by placing each point individually on the ultrasound standard 2D echo images (see FIG. 4G and FIG. 5V), each point representing specific anatomical structures or borders of the specific region of interest of the heart. To aid in such point placement and to speed it up, the present invention provides a feature to allow for all points in one view to be placed simultaneously (semi-automatic, based on A.I. algorithms) rather than being placed manually, one-at-a-time. This is accomplished through the use of overlays or templates (see FIG. 4G and 5V) of the present invention. In a preferred embodiment, the VMS+ software can autonomously build a complete 3D model of structures of the heart, and the entire heart itself, using a reconstruction algorithm employing the 2D ultrasound images (either from 2D echo or 3D echo study) (see FIGS. 6, 7 and 8). In a preferred embodiment, the reconstruction algorithm does this by using a database of MRI wire frames or mesh view (see FIGS. 7A to 7H) as a reference within the software of the present invention such as in U.S. Pat. No. 5,889,524. 3D echocardiogram image data provides tightly spaced images that are acquired to generate a solid volume of image data (see for example, FIGS. 8A to 8H). In a preferred embodiment, the software of the present invention uses algorithms to obtain the best fit to the shape of heart. The input to the algorithm is the points (see for example, FIGS. 7A to 7H and 8A to 8H) placed via the overlays/templates and position and orientation data obtained during the echo study using known patient sensors and transducer sensors (see, for example, U.S. patent application Ser. No. 16/757,755, incorporated herein by reference.).

Another aspect of the present invention is directed to KBR databases. In a preferred embodiment, a KBR database of the present invention is a series of heart shape catalogs. Such catalogs contain data of all four chambers of the heart-atrium and ventricular morphology-in a variety of normal and diseased states. This catalog houses Magnetic Resonance (MR) images representative of the cardiac ventricles and atriums. The algorithm of the present invention requires the user to place anatomic co-ordinates, points or landmarks on the 2D images. These landmarks are used to build an accurate 3D reconstruction shape mesh (defined by vertices, edges and faces) that is unique to that patient for all chambers of the heart (i.e. left ventricle, right ventricle, left atrium, right atrium). See for example, FIGS. 7A to 7H and 8A to 8H. The KBR algorithm can reconstruct the patient's Left Ventricle (LV), Right Ventricle (RV), Left Atrium (LA), and Right Atrium (RA) heart shapes using a limited number of data points selected or entered by the user. The reconstructed shapes are used to calculate the end-diastolic (“ED”) and end-systolic (“ES”) volumes (EDV, ESV), Ejection Fractions (EF), Stroke Volumes and Cardiac Outputs.

In a preferred embodiment, the 2D points placed by the user or via software on the 2D views are translated into 3D using the 3D tracking system data associated with each scan. In the case of 3D Echo, cardiac MRI or cardiac CT, the 3D coordinate system is inherent in the dataset and data from the tracking system is not required. In a preferred embodiment, the VMS+ software of the present invention uses statistical shape analysis to compare the selected points against a database of hearts and returns a precise 3D model of the patient's heart. Based on the structure it is possible to derive the functional aspects of the anatomic structure and whether such function is within normal or diseased parameters.

In a preferred embodiment of the present invention, there is provided a “Guide Panel” or Guide Window” for each chamber of the heart (see 105 of FIG. 1). The Guide Window provides the display of specific protocol images (e.g. templates or overlays with all required anatomic landmarks to be placed on images) of the specific heart chambers. The Guide Panel or Window allows a user to quickly place points according to the points placement guide to ensure that anatomic landmarks needed by the algorithms are dropped on the appropriate images on the appropriate landmarks of the image. Pressing a view button sets the image label to that view. The software of the present invention allows the user to drag and drop a template or overlay from the ED or ES icon with an image label set and without points on the current frame. The template or overlay can be moved, rotated, scaled and then accepted (by double clicking) to place points according to the view and set the phase based on the icon the overlay came from.

Specific protocol images, templates or overlays of the present invention as shown in FIGS. 3A and 3B. For the LV chamber, the software of the present invention can display the Parasternal Long Axis Left Ventricle (PLAX-LV) protocol image (e.g. 301B in FIG. 3B) functioning as a template in the Guide window if the LV tab is selected. The software of the present invention can also display the Parasternal Short Axis Mitral Valve Left Ventricle (PSAXMV-LV) protocol image in the Guide window if the LV tab is selected. The software of the present invention can display the Parasternal Short Axis Mid Left Ventricle (PSAXMID-LV) protocol image (e.g. 303B in FIG. 3B) in the Guide window if the LV tab is selected (as shown in FIG. 4P).

The software of the present invention can display the Parasternal Short Axis Distal Left Ventricle (PSAXDISTAL-LV) protocol image (e.g. 304B in FIG. 3B) in the Guide window if the LV tab is selected.

The software of the present invention can display the Apical Four Chamber Left Ventricle (A4C-LV) protocol image (e.g. 305B in FIG. 3B) in the Guide window if the LV tab is selected.

The software of the present invention can display the Apical Five Chamber Left Ventricle (A5C-LV) protocol image (e.g. 3027B in FIG. 3B) in the Guide window if the LV tab is selected.

The software of the present invention can display the Apical Three Chamber Left Ventricle (A3C-LV) protocol image (e.g. 308B in FIG. 3B) in the Guide window if the LV tab is selected.

The software of the present invention cancan display the Apical Two Chamber Left Ventricle (A2C-LV) protocol image (e.g. 306B in FIG. 3B) in the Guide window if the LV tab is selected.

For the LA chamber, the software of the present invention cancan display the Parasternal Long Axis Left Atrium (PLAX-LA) protocol image (e.g. 317B in FIG. 3B) in the Guide window if the LA tab is selected.

The software of the present invention can display the Apical Four Chamber Left Atrium (A4C-LA) protocol image (e.g. 318B in FIG. 3B) in the Guide window if the LA tab is selected.

The software of the present invention can display the Apical Three Chamber Left Atrium (A3C-LA) protocol image (e.g. 316B in FIG. 3B) in the Guide window if the LA tab is selected.

The software of the present invention can display the Apical Two Chamber Left Atrium (A2C-LA) protocol image (e.g. 319B in FIG. 3B) in the Guide window if the LA tab is selected.

For the RV chamber, the software of the present invention can display the Parasternal Long Axis Right Ventricle (PLAX-RV) protocol image (e.g. 309B in FIG. 3B) in the Guide window if the RV tab is selected.

The software of the present invention can display the Parasternal Right Ventricular Inflow Tract Right Ventricle (PRVIT-RV) protocol image in the Guide window if the RV tab is selected.

The software of the present invention can display the Parasternal Right Ventricular Outflow Tract Right Ventricle (PRVOT-RV) protocol image (e.g. 310B in FIG. 3B) in the Guide window if the RV tab is selected.

The software of the present invention can display the Parasternal Short Axis Aortic Valve Right Ventricle (PSAXAO-RV) protocol image (e.g. 312B in FIG. 3B) in the Guide window if the RV tab is selected.

The software of the present invention can display the Parasternal Short Axis Mid Right Ventricle (PSAXMID-RV) protocol image (e.g. 312B in FIG. 3B) in the Guide window if the RV tab is selected.

The software of the present invention can display the Parasternal Short Axis Distal Right Ventricle (PSAXDISTAL-RV) protocol image in the Guide window if the RV tab is selected.

The software of the present invention can display the Apical Four Chamber Right Ventricle (A4C-RV) protocol image (e.g. 314B in FIG. 3B) in the Guide window if the RV tab is selected.

For the RA chamber, the software of the present invention can display the Parasternal Right Ventricular Inflow Tract Right Atrium (PRVIT-RA) protocol image (e.g. 310B in FIG. 3B) in the Guide window if the RA tab is selected.

The software of the present invention can display the Apical Four Chamber Right Atrium (A4C-RA) protocol image (e.g. 315B in FIG. 3B) in the Guide window if the RA tab is selected.

The software of the present invention can display the Subcostal Inferior Vena Cava Right Atrium (SCIVC-RA) protocol image (e.g. 320B in FIG. 3B) in the Guide window if the RA tab is selected.

For each guide panel, there is a draggable template or overlay with all points to be placed for each view as detailed herein. The user drags the overlay over the 2D image and can adjust it to fit to the image thereby placing the points in the correct spot that corresponds to their anatomical landmark.

As can be seen in FIGS. 4T and 4V, the software of the present invention can display a draggable overlay or template wherein the points correspond to the 4x LV Endocardium anatomical structure points (see 11, 21, 51 and 61 in 306B of FIG. 3B and 280 of FIG. 3A) and 2x Mitral Annulus anatomical structure points (see 41 and 31, in 306B of FIG. 3B and 280 of FIG. 3A) for the Apical Two Chamber for the Left Ventricle (see 306B in FIG. 3B; 280 in FIG. 3A) when the LV tab in the structures table is selected. When an image with A2C-LV is displayed, the user may be able to drag the overlay from the ED (see 460 in FIG. 4T) or the ES (see 450 in FIG. 4T) icon to the 2D image and release in position on the 2D image. Similarly, the software of the present invention can display a draggable overlay of the 4x LV Endocardium anatomical structure points for the Parasternal Short Axis Mid for the Left Ventricle when the LV tab in the structures table is selected (see FIG. 4G). When an image with PSAXMID-LV is displayed, the user may be able to drag the overlay from the ED or the ES icon to the 2D image and release in position on the 2D image. Similarly, the software of the present invention may display a draggable overlay of the 4x LV Endocardium anatomical structure points for the Parasternal Short Axis Distal for the Left Ventricle when the LV tab in the structures table is selected.

When an image with PSAXDISTAL-LV is displayed, the user may be able to drag the overlay from the ED or the ES icon to the 2D image and release in position on the 2D image.

The software of the present invention may display a draggable overlay of the 1x Apex, 4x LV Endocardium, 2x Mitral Annulus anatomical structure points for the Apical 4 Chamber for Left Ventricle when the LV tab in the structures table is selected. When an image with A4C-LV is displayed, the user may be able to drag the overlay from the ED or the ES icon to the 2D image and release in position on the 2D image.

The software of the present invention may display a draggable overlay of the 4x LV Endocardium, 2x Aortic Annulus anatomical structure points for the Apical 5 Chamber for Left Ventricle when the LV tab in the structures table is selected. When an image with A5C-LV is displayed, the user may be able to drag the overlay from the ED or the ES icon to the 2D image and release in position on the 2D image.

The software of the present invention can display a draggable overlay of the 4x LV Endocardium, 2x Aortic Annulus, and 2x Mitral Annulus anatomical structure points for the Apical 3 Chamber for Left Ventricle when the LV tab in the structures table is selected. When an image with A3C-LV is displayed, the user can be able to drag the overlay from the ED or the ES icon to the 2D image and release in position on the 2D image.

The software of the present invention can display a draggable overlay of the 4x LV Endocardium and 2x Mitral Annulus anatomical structure points for the Apical 2 Chamber for Left Ventricle when the LV tab in the structures table is selected. When an image with A2C-LV is displayed, the user can be able to drag the overlay from the ED or the ES icon to the 2D image and release in position on the 2D image.

The software of the present invention can display a draggable overlay of the 3x LA Endocardium, 2x Mitral Annulus anatomical structure points for the Parasternal Long Axis for Left Atrium when the LA tab in the structures table is selected. When an image with PLAX-LA is displayed, the user can drag and drop the template or overlay from the ED or the ES icon to the 2D image and release in position on the 2D image.

The software of the present invention may display a draggable overlay of the 4x LA Endocardium, 2x Mitral Annulus anatomical structure points for the Apical 4 Chamber for Left Atrium when the LA tab in the structures table is selected. When an image with A4C-LA is displayed, the user can drag and drop the overlay from the ED or the ES icon to the 2D image and release in position on the 2D image.

The software of the present invention can display a draggable overlay of the 4x LA Endocardium and 2x Mitral Annulus anatomical structure points for the Apical 3 Chamber for Left Atrium when the LA tab in the structures table is selected. When an image with A3C-LA is displayed, the user can be able to drag the overlay from the ED or the ES icon to the 2D image and release in position on the 2D image.

The software of the present invention can display a draggable overlay of the 4x LA Endocardium and 2x Mitral Annulus anatomical structure points for the Apical 2 Chamber for Left Atrium when the LA tab in the structures table is selected. When an image with A2C-LA is displayed, the user can be able to drag the overlay from the ED or the ES icon to the 2D image and release in position on the 2D image.

The software of the present invention can display a draggable overlay of the 3x RV Endocardium and 2x RV Septum anatomical structure points for the Parasternal Long Axis for the Right Ventricle when the RV tab in the structures table is selected. When an image with PLAX-RV is displayed, the user can be able to drag the overlay from the ED or the ES icon to the 2D image and release in position on the 2D image.

The software of the present invention can display a draggable overlay of the 3x RV Endocardium and 2x Tricuspid Annulus anatomical structure points for the Parasternal Right Ventricular Inflow Tract for Right Ventricle when the RV tab in the structures table is selected. When an image with PRVIT-RV is displayed, the user can be able to drag the overlay from the ED or the ES icon to the 2D image and release in position on the 2D image.

The software of the present invention can display a draggable overlay of the 1x RV Endocardium, 1x RV Septum, 2x Pulmonic Annulus anatomical structure points for the Parasternal Right Ventricular Outflow Tract for Right Ventricle when the RV tab in the structures table is selected. When an image with PRVOT-RV is displayed, the user can be able to drag the overlay from the ED or the ES icon to the 2D image and release in position on the 2D image.

The software of the present invention can display a draggable overlay of the 2x RV Endocardium, 1x Conal Septum, 2x Pulmonic Annulus, 2x Tricuspid Annulus anatomical structure points for the Parasternal Short Axis Aortic Outflow Tract for Right Ventricle when the RV tab in the structures table is selected. When an image with PSAXAO-RV is displayed, the user can be able to drag the overlay from the ED or the ES icon to the 2D image and release in position on the 2D image.

The software of the present invention can display a draggable overlay of the 2x RV Endocardium, 2x RV Septal Edge, and 1x RV Septum anatomical structure points for the Parasternal Short Axis Mid for Right Ventricle when the RV tab in the structures table is selected. When an image with PSAXMID-RV is displayed, the user may be able to drag the overlay from the ED or the ES icon to the 2D image and release in position on the 2D image.

The software of the present invention can display a draggable overlay of the 2x RV Septal Edge anatomical structure points for the Parasternal Short Axis Distal for Right Ventricle when the RV tab in the structures table is selected. When an image with PSAXDISTAL-RV is displayed, the user can be able to drag the overlay from the ED or the ES icon to the 2D image and release in position on the 2D image.

The software of the present invention can display a draggable overlay of the 2x RV Endocardium, 2x Tricuspid Annulus, 2x RV Septum, 1x Apex, and 1x Basal Bulge anatomical structure points for the Apical 4 Chamber for Right Ventricle when the RV tab in the structures table is selected. When an image with A4C-RV is displayed, the user can be able to drag the overlay from the ED or the ES icon to the 2D image and release in position on the 2D image.

The software of the present invention can display a draggable overlay of the 3x RA Endocardium and 2x Tricuspid Annulus anatomical structure points for the Parasternal Right Ventricular Inflow Tract for Right Atrium when the RA tab in the structures table is selected. When an image with PRVIT-RA is displayed, the user can be able to drag the overlay from the ED or the ES icon to the 2D image and release in position on the 2D image.

The software of the present invention can display a draggable overlay of the 4x RA Endocardium and 2x Tricuspid Annulus anatomical structure points for the Apical Four Chamber for Right Atrium when the RA tab in the structures table is selected. When an image with A4C-RA is displayed, the user can be able to drag the overlay from the ED or the ES icon to the 2D image and release in position on the 2D image.

The software of the present invention can display a draggable overlay of the 3x RA Endocardium anatomical structure points for Subcostal Inferior Vena Cava for Right Atrium when the RA tab in the structures table is selected. When an image with SCIVC-RA is displayed, the user can be able to drag the overlay from the ED or the ES icon to the 2D image and release in position on the 2D image.

The software of the present invention may allow the overlay for each anatomical view to be scaled in size when aligning the anatomic points with the anatomy of the 2D image.

In a preferable embodiment, the software of the present invention may also provide the ability to growing and/or shrink the overlays. For example, there is a cursor 401 (e.g. in a preferred embodiment, a red circle) on the overlays that does not scale with the overlay. Clicking inside the circle allows for moving the overlay (see FIG. 4W). Clicking outside the circle allows for scaling the overlay.

The software of the present invention can allow the overlay for each anatomical view to be rotated when aligning the anatomic points with the anatomy of the 2D image. (see FIG. 4V)

This includes each anatomical view for each chamber (i.e. LV, RV, LA, RA). The software of the present invention can snap the points placed at the points shown on the overlay to the image border. ED and ES will be set by the placement of the overlay after scaling and positioning. ED or ES is chosen depending on if the overlay was dragged from the ED or the ES icon.

The foregoing description has been presented for the purpose of illustration and maybe not intended to be exhaustive or to limit the invention to the precise form disclosed. Other modifications, variations and alterations are possible in light of the above teaching and may be apparent to those skilled in the art, and may be used in the design and manufacture of other embodiments according to the present invention. It may be intended the scope of the invention be limited not by this description but only by the claims forming a part of this application and/or any patent issuing therefrom.

1. A method for using a computer to reconstructing anatomical features or an organ, comprising the steps of:
(a) obtaining 3D image data from imaging the organ;
(b) obtaining a 2D slice of the organ from the 3D image data and optimizing fit of a template having predetermined landmark points to the 2D slice so that a plurality of anatomical features of the 2D slice are generally aligned with the corresponding landmark points of the template;
(c) assigning each point of the template to the corresponding anatomical feature of the 2D slice based on the fit of the template; and
(d) repeating steps (b) and (c) to produce a functional reconstruction of the organ.

2. The method of claim 1 wherein the 3D image data is echo-based or MRI 3D datasets.

3. The method of claim 2 wherein the organ is a heart.

4. The method of clam 3 wherein step (d) employs a KBR algorithm.

5. The method of claim 4 wherein the templates are selected based on the anatomical regions of the heart.

6. The method of claim 5 wherein the KBR algorithm functionally reconstructs the Left Ventricle (LV), Right Ventricle (RV), Left Atrium (LA), and Right Atrium (RA) of the using the assigned points of the template.

7. The method of claim 6 wherein each point of the template is assigned automatically.

8. The method of claim 6 wherein each point of the template is assigned by the user.

9. The method of claim 6 wherein the template corresponds to anatomic region of the heart.

10. The method of claim 9 wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

11. A system for producing a functional reconstruction of the organ, comprising:
(a) an imaging system for producing 3D image data of the organ;
(b) a memory for storing:
(i) the 3D image data; and
(ii) machine instructions that define steps for processing the data derived from the 3D image data; and
(c) a processor that is coupled to the memory, said processor executing the machine instructions, causing the processor to:
(i) obtain a 2D slice of the organ from the 3D image data and optimizing fit of the template to the 2D slice so that a plurality of anatomical features of the 2D slice are generally aligned with the corresponding points of the template;
(ii) assign each point of the template to the corresponding anatomical feature of the 2D slice based on the fit of the template; and
(iii) repeating steps (ii) and (iii) to produce a functional reconstruction of the organ.

12. The system of claim 11 wherein the 3D image data is echo-based or MRI 3D datasets.

13. The system of claim 12 wherein the organ is a heart.

14. The system of clam 13 wherein step (c)(iii) employs a KBR algorithm.

15. The system of claim 14 wherein the templates are selected based on the anatomical regions of the heart.

16. The system of claim 15 wherein the KBR algorithm functionally reconstructs the Left Ventricle (LV), Right Ventricle (RV), Left Atrium (LA), and Right Atrium (RA) of the using the assigned points of the template.

17. The system of claim 16 wherein each point of the template is assigned automatically.

18. The system of claim 16 wherein each point of the template is assigned by the user.

19. The system of claim 16 wherein the template corresponds to anatomic region of the heart.

20. The system of claim 19 wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

21. A method for calculating, analyzing and displaying anatomical features or an organ, comprising the steps of:
(a) selecting 3D image data from imaging the organ;
(b) obtaining a plurality of 2D slices of the organ from the 3D image data, the plurality of 2D slices corresponding to one of the XY, YZ and/or XZ plane of the organ; and
(c) dynamically displaying the plurality of 2D slices in one of the XY, YZ and XZ planes orthogonally and having a user view the plurality of 2D slices in each of the XY, YZ or XZ planes

22. The method of claim 21 wherein the 3D image data is echo-based or MRI 3D datasets.

23. The method of claim 22 wherein the organ is a heart.

24. The method of claim 23 further comprising optimizing fit of a template to the 2D slice so that a plurality of anatomical features of the 2D slice are generally aligned with the corresponding points of the template.

25. The method of claim 24 wherein the templates are selected based on the anatomical regions of the heart.

26. The method of claim 25 wherein each point of the template is assigned automatically.

27. The method of claim 26 wherein each point of the template is assigned by the user.

28. The method of claim 25 wherein the template corresponds to anatomic region of the heart.

29. The method of claim 28 wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

30. A method for using a computer to reconstructing anatomical features or an organ, comprising the step of creating a 2D slice having predetermined landmark points generally corresponding with selected anatomical features of the organ from 3D image data.

31. The method of claim 30 wherein the 3D image data is echo-based or MRI 3D datasets.

32. The method of claim 31 wherein the organ is a heart.

33. The method of claim 32 wherein the templates are selected based on the anatomical regions of the heart.

34. The method of claim 33 wherein the template is selected from the group consisting of Parasternal Long Axis Left Ventricle (PLAX-LV), Parasternal Short Axis Mitral Valve Left Ventricle (PSAXMV-LV), Parasternal Short Axis Mid Left Ventricle (PSAXMID-LV), Parasternal Short Axis Distal Left Ventricle (PSAXDISTAL-LV), Apical Four Chamber Left Ventricle (A4C-LV), Apical Five Chamber Left Ventricle (A5C-LV), Apical Three Chamber Left Ventricle (A3C-LV), Apical Two Chamber Left Ventricle (A2C-LV), Parasternal Long Axis Left Atrium (PLAX-LA), Apical Four Chamber Left Atrium (A4C-LA), Apical Three Chamber Left Atrium (A3C-LA), Apical Two Chamber Left Atrium (A2C-LA), Parasternal Long Axis Right Ventricle (PLAX-RV), Parasternal Right Ventricular Inflow Tract Right Ventricle (PRVIT-RV), Parasternal Right Ventricular Outflow Tract Right Ventricle (PRVOT-RV), Parasternal Short Axis Aortic Valve Right Ventricle (PSAXAO-RV), Parasternal Short Axis Mid Right Ventricle (PSAXMID-RV), Parasternal Short Axis Distal Right Ventricle (PSAXDISTAL-RV), Apical Four Chamber Right Ventricle (A4C-RV), Parasternal Right Ventricular Inflow Tract Right Atrium (PRVIT-RA), Apical Four Chamber Right Atrium (A4C-RA), and Subcostal Inferior Vena Cava Right Atrium (SCIVC-RA).

35. A graphical user interface on an electronic device with a screen display, the graphic user interface comprising:
(a) A pointer displayed on the screen display, the movements of the pointer on the display screen controlled by a user;
(b) a first area of the screen displaying a plurality of 2D slices of the organ obtained from 3D image data from imaging an organ, the plurality of 2D slices corresponding to one of the XY, YZ and/or XZ plane of the organ;
(c) a second area of the screen separate from the first area that dynamically displays the plurality of 2D slices in each of the XY, YZ and XZ planes orthogonally;
wherein based on the movements of the pointer the user can change the plurality of 2D slices that are displayed.

36. The graphic user interface of claim 35 wherein the 3D image data is echo-based or MRI 3D datasets.

37. The graphic user interface of claim 36 wherein the organ is a heart.
